(19) United States
(12) Patent Application Publication
(10) Pub. No.: US 2025/0388964 A1
(43) Pub. Date: Dec. 25, 2025

(54) MULTI-SPECIES CHIP TO DETECT DNA-METHYLATION

(71) Applicants: Evonik Operations GmbH, Essen (DE); Deutsches Krebsforschungszentrum Stiftung des Öffentlichen Rechts, Heidelberg (DE)

(72) Inventors: Sanjanaa NAGARAJAN, Singapore (SG); Suki ROY, Sydney (AU); Florian BÖHL, Neckargemünd (DE); Rose WHELAN, Ambrosden Oxfordshire (GB); Lingzhi HUANG, Singapore (SG); Kit Yeng WONG, Singapore (SG); Sarah CHAN, Bellevue, WA (US); Daniel FRANKE, Essen (DE); Frank LYKO, Heidelberg (DE); Geetha VENKATESH, Heidelberg (DE); Günter RADDATZ, Heidelberg (DE); Sina LYKO-TÖNGES, Heidelberg (DE); Walter PFEFFERLE, Langgöns (DE); Rainer WESSEL, Heidelberg (DE)

(73) Assignees: Evonik Operations GmbH, Essen (DE); Deutsches Krebsforschungszentrum Stiftung des Öffentlichen Rechts, Heidelberg (DE)

(21) Appl. No.: 19/107,634

(22) PCT Filed: Aug. 23, 2023
(86) PCT No.: PCT/EP2023/073123

§ 371 Date: Feb. 28, 2025

(30) Foreign Application Priority Data
Sep. 1, 2022 (EP) 22193451.6

(51) Int. Cl. C12Q 1/6876

(52) U.S. Cl. CPC C12Q 1/6876; C12Q 2600/156

(57) ABSTRACT

A DNA methylation-based array includes at least: a first plurality of distinct locations, each location having at least one probe molecule comprising a nucleic acid sequence complementary to a CpG site from a first plurality of CpG sites of a first animal species; and a second plurality of distinct locations, each location having at least one probe molecule comprising a nucleic acid sequence complementary to a CpG site from a second plurality of CpG sites of a second animal species, wherein the first and second animal species are each independently selected from the group consisting of virus, mammals, birds and aquatic animals, and wherein the first plurality of CpG sites comprises at least 1000 CpG sites of the first animal species; and the second plurality of CpG sites comprises at least 1000 CpG sites of the second animal species.

### CROSS REFERENCE TO RELATED APPLICATIONS

This application is a National Stage of International Application No. PCT/EP2023/073123 filed Aug. 23, 2023, claiming priority based on European Patent Application No. 22193451.6 filed Sep. 1, 2022.

### FIELD OF THE INVENTION

The present invention relates to a multi-species chip. In particular, the multi-species chip is a methylation-based array that comprises probes complementary to nucleic acids of CpG sites distinct to more than one animal species. The animal species may be from any class of animals selected from invertebrates and vertebrates where the vertebrates may be mammals, birds, fish or the like.

### BACKGROUND OF THE INVENTION

Epigenetics is the study of inherited traits caused by mechanisms other than changes in the underlying DNA sequence. In other words, epigenetic marks “orchestrate” our genes. Epigenetic marks can be either chemical (e.g. methylation), protein-based (e.g. histones) or a combination of the two. During development and cell differentiation, DNA methylation is dynamic, but some DNA methylation patterns may be retained as a form of epigenetic memory, accumulated and/or inherited to next generation. Those changes might be responsible for heritable changes in gene activity as DNA methylation events have been shown to be regulation mechanisms associated with gene silencing, expression, chromatin remodelling or imprinting. Epigenetics is attractive for animal breeding as it may identify causality and heritability of complex traits and diseases. DNA methylation patterns are modified along the life of an individual by environmental forces like diet, stress, drugs, or pollution among many others. Some environments are more likely to increase certain methylation patterns, and these patterns could contribute to the epigenetic and/or phenotypic variation between individuals.

Epigenetics technologies may therefore be used for example to study correlations between the epigenome and specific phenotypes or to develop compound biomarkers for differentiation of different environmental treatment groups as is currently being done in the Epigenome-Wide Association Studies (EWAS).

Traditionally, global methylation patterns especially for non-human species have been assessed from extracted DNA from different tissue and/or cells, by using whole genome bisulfite sequencing (WGBS) or reduced representation bisulfite sequencing (RRBS). Both approaches first use a bisulfite treatment step to convert all unmethylated cytosine nucleotides in the genome to uracil, leaving methylated and hemi-methylated cytosine nucleotides unchanged (Stevens et al., 2013).

Next generation sequencing is performed, and sequences generated are processed (aligned to reference genomes) and analysed to indicate methylation differences at individual CpG sites. WGBS covers the CpG sites on the whole genome, while RRBS covers only 3-4% of all methylation sites of a genome but represents 85% of CpG sites of the dynamically methylated regions (Illumina, Field Guide to Methylation Methods, 2016). These technologies while highly informative are costly, time-consuming, and computationally intensive, prohibiting fast turnaround times.

DNA-Methylation-based arrays allow for a high-throughput and robust method to determine semi-quantitative/quantitative DNA-methylation information through a small sample of extracted DNA of interest. These custom designed arrays use Illumina iScan and Infinium platform technology, which allows on each chip for example hundreds of thousands of different bead types that covalently bind DNA-methylation probes. Each probe represents one CpG Methylation site at the end of the probe sequence. DNA samples undergo bisulfite conversion, amplification, fragmentation, precipitation and resuspension steps before hybridization on an Illumina Infinium array chip. Once on the chip the DNA hybridizes to the beads for each CpG site so that methylation statues at each site can be detected specifically through single nucleotide extension.

To date, DNA methylation-based arrays are available for a limited number of species of high research relevance (e.g. mouse, human) and most arrays are only usable for the single specific species they were designed for. While a few arrays have been designed for use with multiple species, those that exist use CpG sites that are conserved for a subset of animals (e.g. class Mammalia) (Arneson, A., et al, Nat Commun 13, 783 (2022)). With this design, CpG sites cannot be added unless they are conserved among the entire class, regardless of whether they are highly relevant and interesting for evaluation in one or a few of the species represented. Additionally, this method restricts design to similar categorizations of organisms.

There is thus still a lack of methylation-based arrays that contain probes that are specific for multiple species and that makes the process of detecting methylation changes in DNA cost-effective, robust, reliable, and efficient.

### BRIEF DESCRIPTION OF FIGURES

FIG. 1 refers to a design of the array with three different animal species, Chinese Hamster Ovary cell lines (CHO), chicken and crayfish. The % shows the % of CpG sites found in the array that belong to a particular species. For example, 16% of the total CpG sites are from crayfish, 36% from CHO cells and 48% from chicken.

FIG. 2 is a distribution plot of the mean beta value for the titrated chicken samples of Example 3.

FIG. 3 is a distribution plot of the mean beta value for the titrated CHO samples of Example 4.

### DESCRIPTION OF THE INVENTION

The present invention solves the problems above by providing a methylation-based array that contains probes for multiple species, where the probes are specific for CpG targets that are found on each individual species on the multi-species chip. This is especially advantageous as the results of the methylation-based array is accurate and reproducible. In particular, the probes on the multi-species chip according to any aspect of the present invention is specific for CpG targets from the different species that are not conserved among all species represented on the chip and/or the probes are designed not only for different purposes in the different species represented on the multi-species chip, but they are also new and specific. Further the multi-species chip according to any aspect of the present invention includes species from different classes of animals (mammals, vertebrates, invertebrates) together on a single array on a chip comprising one or more (e.g. 12, 24, 48, or 96) arrays. This offers the flexibility of generating data from multiple samples either from single species or several species simultaneously in a much faster and cost-efficient way.

The multi-species chip according to any aspect of the present invention would also improve cost-savings, flexibility and efficiency for research labs which particularly work on a variety of species by allowing the labs to save the time and energy that goes into developing and stocking multiple chips and waiting for sufficient samples of each individual species to be available to run a full chip for the most cost-effective analytics or using traditional sequencing technologies.

Further, compared to traditional sequencing which can take weeks to generate data, the array technology has a much shorter turn-around time. The volume and complexity of data generated is lesser compared to sequencing making it computationally less intensive. This allows for quicker computation to achieve interpretable results from experimental groups. Overall microarray technology is roughly 10× faster and 10× cheaper than traditional sequencing while still quantifiable for the methylation level at specific CpG sites. Methylation-array technology therefore offers a fast & flexible system that can be used for many applications, allowing for the scalability of epigenetics research, and commercialization of DNA-methylation based solutions for along the food value chains.

The term ‘epigenetic change’ as used herein refers to a chemical (e.g., methylation) change or protein (e.g., histones) change that takes place to a gene body or a promoter thereof. Through epigenetic changes, environmental factors like. diet, stress and prenatal nutrition can make an imprint on genes passed from one generation to the next.

According to one aspect of the present invention, there is provided a DNA methylation-based array comprising at least:
a first plurality of distinct locations, each location having at least one probe molecule comprising a nucleic acid sequence complementary to a CpG site from a first plurality of CpG sites of a first animal species; and
a second plurality of distinct locations, each location having at least one probe molecule comprising a nucleic acid sequence complementary to a CpG site from a second plurality of CpG sites of a second animal species
   wherein the first and second animal species are each independently selected from the group consisting of virus, mammals, birds and aquatic animals, and
the mammal is at least one livestock or animal cell line;
the bird is at least one poultry; and
the aquatic animal is at least one crustacean, cephalopod or fish,
and
   wherein the first plurality of CpG sites comprises at least 1000 CpG sites of the first animal species; and the second plurality of CpG sites comprises at least 1000 CpG sites of the second animal species.

The array according to any aspect of the present invention is especially advantageous as the CpG sites to which the nucleic acid sequences of the array bind to, are specific and distinct to the first or second animal species and are not generic or universal to different species. This makes the array according to any aspect of the present invention, accurate and efficient at identifying not only the species of animal being tested but also other features of the test animal, like the epigenetic age, geographic origin, whether the animal has been exposed to antibiotics and/or veterinary chemicals, whether the test animal has been bred under specific conditions and the like.

The term “array” as used herein refers to an intentionally created collection of probe molecules which can be prepared either synthetically or biosynthetically. The probe molecules in the array can be identical or different from each other. The array can assume a variety of formats, for example, libraries of soluble molecules; libraries of compounds tethered to resin beads, silica chips, or other solid supports.

In particular, a DNA methylation-based array provides a convenient platform for simultaneous analysis of large numbers of CpG sites, for example, at least 2, 3, 4, 5, 6, 7, 8, 9, 10, 50, 100, 500, 1000, 5000, 10,000, 100,000 or more sites or loci. In particular, the array comprises a plurality of different probe molecules that can be attached to a substrate or otherwise spatially distinguished in an array. Examples of arrays that may be used according to any aspect of the present invention include slide arrays, silicon wafer arrays, liquid arrays, bead-based arrays and the like. In one example, array technology used according to any aspect of the present invention combines a miniaturized array platform, a high level of assay multiplexing, and scalable automation for sample handling and data processing.

In particular, the array according to any aspect of the present invention may be an array of arrays, also referred to as a composite array, having a plurality of individual arrays that is configured to allow processing of multiple samples simultaneously. Examples of composite arrays and the technology behind them are disclosed at least in U.S. Pat. No. 6,429,027 and US 2002/0102578. A substrate of a composite array may include a plurality of individual array locations, each having a plurality of probes, and each physically separated from other assay locations on the same substrate such that a fluid contacting one array location is prevented from contacting another array location. Each array location can have a plurality of different probe molecules that are directly attached to the substrate or that are attached to the substrate via rigid particles in wells (also referred to herein as beads in wells).

In one example, an array substrate can be a fibre optical bundle or array of bundles as described in U.S. Pat. Nos. 6,023,540, 6,200,737 and/or 6,327,410. An optical fibre bundle or array of bundles can have probes attached directly to the fibres or via beads. A skilled person would be able to easily determine which substrate will be most suitable for the array according to any aspect of the present invention. WO2004110246 further discloses other substrates and methods of attaching beads to the substrates that may be used in the array according to any aspect of the present invention.

In one example, a surface of the substrate may have physical alterations to enable the attachment of probes or produce array locations. For example, the surface of a substrate can be modified to contain chemically modified sites that are useful for attaching, either-covalently or non-covalently, probe molecules or particles having attached probe molecules. Probes may be attached using any of a variety of methods known in the art including, an ink-jet printing method, a spotting technique, a photolithographic synthesis method, or printing method utilizing a mask. WO2004110246 discloses these techniques in more detail.

In one example, the DNA methylation-based array according to any aspect of the present invention may be a bead-based array, where the beads are associated with a solid support such as those commercially available from Illumina, Inc. (San Diego, Calif.). An array of beads useful according to any aspect of the present invention can also be in a fluid format such as a fluid stream of a flow cytometer or similar device. Commercially available fluid formats for distinguishing beads include, for example, those used in XMAP™ technologies from Luminex or MPSS™ methods from Lynx Therapeutics.

The term “solid support”, “support”, and “substrate” as used herein are used interchangeably and refer to a material or group of materials having a rigid or semi-rigid surface or surfaces. In many examples, at least one surface of the solid support will be substantially flat, although in some examples it may be desirable to physically separate synthesis regions for different compounds with, for example, wells, raised regions, pins, etched trenches, or the like.

The DNA methylation array according to any aspect of the present invention may be a very high-density array, for example, those having from about 10,000,000 probes/cm² to about 2,000,000,000 probes/cm² or from about 100,000,000 probes/cm² to about 1,000,000,000 probes/cm². High density arrays are especially useful according to any aspect of the present invention for including the multitude of CpG sites from the different species on the array.

The DNA methylation array according to any aspect of the present invention may be used to analyse or evaluate such pluralities of loci simultaneously or sequentially as desired. In one example, a plurality of different probe molecules can be attached to a substrate or otherwise spatially distinguished in an array. Each probe is typically specific for a particular locus and can be used to distinguish methylation state of the locus.

The array according to any aspect of the present invention comprises:
a first plurality of distinct locations, each location having at least one probe molecule comprising a nucleic acid sequence complementary to a CpG site from a first plurality of CpG sites of a first animal species and the first plurality of CpG sites comprises at least 1000 CpG sites of the first animal species; and
a second plurality of distinct locations, each location having at least one probe molecule comprising a nucleic acid sequence complementary to a CpG site from a second plurality of CpG sites of a second animal species and the second plurality of CpG sites comprises at least 1000 CpG sites of the second animal species.

The term “probe molecules” as used herein refers to a surface-immobilized molecule that can be recognized by a particular target. Probes used in the array can be specific for the methylated allele of a CpG site, the non-methylated allele of the CpG site or both.

The term “target” as used herein refers to a molecule that has an affinity for a given probe molecule. Targets may be naturally occurring or man-made molecules. Also, they can be employed in their unaltered state or as aggregates with other species. Targets may be attached, covalently or noncovalently, to a binding member, either directly or via a specific binding substance. Examples of targets which can be employed according to any aspect of the present invention are methylated and non-methylated CpG sites. Targets are sometimes referred to in the art as anti-probes. As the term targets is used herein, no difference in meaning is intended.

In particular, the probe molecule according to any aspect of the present invention comprises a nucleic acid sequence that is complementary to a distinct CpG site of a first animal species. The array according to any aspect of the present invention thus comprises several distinct or unique locations, wherein each location comprises a specific probe molecule that is complementary to a distinct CpG site of an animal species. The array thus comprises a plurality of locations, each location with a specific probe molecule that is complementary to a distinct CpG site of an animal species. In particular, the array according to any aspect of the present invention, comprises distinct locations, where each location comprises a specific probe molecule that is complementary to a distinct CpG site of at least two animal species. The array according to any aspect of the present invention thus comprises distinct locations with specific probe molecules where each probe molecule is complementary to a distinct CpG site from at least two animal species.

In particular, the plurality of CpG sites of each animal species comprises at least about 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, or 10000 CpG sites. More in particular, the first plurality of CpG sites comprises at least 1000 CpG sites of the first animal species and the second plurality of CpG sites comprises at least 1000 CpG sites of the second animal species.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Perfectly complementary refers to 100% complementarity over the length of a sequence. For example, a 25 base probe is perfectly complementary to a target when all 25 bases of the probe are complementary to a contiguous 25 base sequence of the target with no mismatches between the probe and the target over the length of the probe.

As used herein, a “CpG site” or “methylation site” is a nucleotide within a nucleic acid (DNA or RNA) that is susceptible to methylation either by natural occurring events in vivo or by an event instituted to chemically methylate the nucleotide in vitro. Some of these sites may be hypermethylated and some may be hypomethylated in a cell.

As used herein, a “methylated nucleic acid molecule” refers to a nucleic acid molecule that contains one or more nucleotides that is/are methylated.

A “CpG island” as used herein describes a segment of DNA sequence that comprises a functionally or structurally deviated CpG density. For example, Yamada et al. have described a set of standards for determining a CpG island: it must be at least 400 nucleotides in length, has a greater than 50% GC content, and an OCF/ECF ratio greater than 0.6 (Yamada et al., 2004, Genome Research, 14, 247-266). Others have defined a CpG island less stringently as a sequence at least 200 nucleotides in length, having a greater than 50% GC content, and an OCF/ECF ratio greater than 0.6 (Takai et al., 2002, Proc. Natl. Acad. Sci. USA, 99, 3740-3745). In context of the present invention, the terms “methylation profile”, “methylation pattern”, “methylation state” or “methylation status,” are used herein to describe the state, situation or condition of methylation of a genomic sequence, and such terms refer to the characteristics of a DNA segment at a particular genomic locus in relation to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles.

The term “methylation status” refers to the status of a specific methylation site (i.e. methylated vs. non-methylated) which means a residue or methylation site is methylated or not methylated. Then, based on the methylation status of one or more methylation sites, a methylation profile may be determined.

The term “methylation level” refers to the level of a specific methylation site which can range from 0 (=unmethylated) to 1 (=fully methylated). Thus, based on the methylation level of one or more methylation sites, a methylation profile may be determined. Accordingly, the term “methylation” profile” or also “methylation pattern” refers to the relative or absolute concentration of methylated C or unmethylated C at any particular stretch of residues in a biological sample. For example, if cytosine (C) residue(s) not typically methylated within a DNA sequence are more methylated in a sample, it may be referred to as “hypermethylated”; whereas if cytosine (C) residue(s) typically methylated within a DNA sequence are less methylated, it may be referred to as “hypomethylated”. Likewise, if the cytosine (C) residue(s) within a DNA sequence (e.g., sample nucleic acid) are more methylated when compared to another sequence from a different region or from a different individual (e.g., relative to normal nucleic acid), that sequence is considered hypermethylated compared to the other sequence. Alternatively, if the cytosine (C) residue(s) within a DNA sequence are less methylated as compared to another sequence from a different region or from a different individual, that sequence is considered hypomethylated compared to the other sequence. These sequences are said to be “differentially methylated”. For example, when the methylation status differs between inflamed and non-inflamed tissues, the sequences are considered “differentially methylated”. Measurement of the levels of differential methylation may be done by a variety of ways known to those skilled in the art. One method is to measure the methylation level of individual interrogated CpG sites determined by the bisulfite sequencing method, as a non-limiting example.

As used herein, the term “genomic material” refers to nucleic acid molecules or fragments of the genome of the animal according to any aspect of the present invention. In particular, such nucleic acid molecules or fragments are DNA or RNA or hybrids thereof, and most preferably are molecules of the DNA genome of a subject or group of subjects.

The term ‘biological sample’ as used herein may be selected from the group consisting of muscle, organ tissue, milk, blood, brain, sperm and any other tissue or sample that provides genomic DNA to be used in the method according to any aspect of the present invention. In particular, the biological sample may comprise any biological material obtained from the subject that contains

DNA, and may be liquid, solid or both, may be tissue or bone, or a body fluid such as blood, lymph, etc. In particular, the biological sample useful for the present invention may comprise biological cells or fragments thereof.

As used herein, the “DNA sample” refers to the DNA extracted from a cell of the animal according to any aspect of the present invention using known methods in the art.

‘Bisulfite treatment’ of genomic DNA used interchangeably with the term ‘bisulfite modification’, refers to the treatment of the genomic DNA with a deaminating agent such as a bisulfite that may be used to treat all DNA, methylated or not. In particular, the term “bisulfite” as used herein encompasses any suitable type of bisulfite, such as sodium bisulfite, or other chemical agents that are capable of chemically converting a cytosine (C) to an uracil (U) without chemically modifying a methylated cytosine and therefore can be used to differentially modify a DNA sequence based on the methylation status of the DNA, e.g., U.S. Pat. Pub. US 2010/0112595. As used herein, a reagent that “differentially modifies” methylated or non-methylated DNA encompasses any reagent that modifies methylated and/or unmethylated DNA in a process through which distinguishable products result from methylated and non-methylated DNA, thereby allowing the identification of the DNA methylation status. Such processes may include, but are not limited to, chemical reactions (such as a C to U conversion by bisulfite) and enzymatic treatment (such as cleavage by a methylation-dependent endonuclease). Thus, an enzyme that preferentially cleaves or digests methylated DNA is one capable of cleaving or digesting a DNA molecule at a much higher efficiency when the DNA is methylated, whereas an enzyme that preferentially cleaves or digests unmethylated DNA exhibits a significantly higher efficiency when the DNA is not methylated.

An alternative method available in the art may be used instead of bisulfite treatment. A skilled person will understand which other methods to use. In one example, TET-assisted pyridine borane sequencing (TAPS) may be used for detection of 5mC and 5hmC (Yibin Liu, et al., Nature Biotechnology, 37:424-429 (2019).

As used herein, a “methylated nucleotide” or a “methylated nucleotide base” refers to the presence of a methyl moiety on a nucleotide base, where the methyl moiety is usually not present in a recognized typical nucleotide base. For example, cytosine in its usual form does not contain a methyl moiety on its pyrimidine ring, but 5-methylcytosine contains a methyl moiety at position 5 of its pyrimidine ring. Therefore, cytosine in its usual form may not be considered a methylated nucleotide and 5-methylcytosine may be considered a methylated nucleotide. In another example, thymine may contain a methyl moiety at position 5 of its pyrimidine ring, however, for purposes herein, thymine may not be considered a methylated nucleotide when present in DNA. Typical nucleotide bases for DNA are thymine, adenine, cytosine and guanine. Typical bases for RNA are uracil, adenine, cytosine and guanine. Correspondingly a “methylation site” is the location in the target gene nucleic acid region where methylation has the possibility of occurring. For example, a location containing CpG is a methylation site wherein the cytosine may or may not be methylated. In particular, the term “methylated nucleotide” refers to nucleotides that carry a methyl group attached to a position of a nucleotide that is accessible for methylation. These methylated nucleotides are usually found in nature and to date, methylated cytosine that occurs mostly in the context of the dinucleotide CpG, but also in the context of CpNpG- and CpNpN-sequences may be considered the most common. In principle, other naturally occurring nucleotides may also be methylated but they will not be taken into consideration with regard to any aspect of the present invention.

In context of the present invention, the terms “methylation profile”, “methylation pattern”, “methylation state” or “methylation status,” are used herein to describe the state, situation or condition of methylation of a genomic sequence, and such terms refer to the characteristics of a DNA segment at a particular genomic locus in relation to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles.

The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample.

The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

In particular, the first and second animal species are selected from the group consisting of virus, mammals, birds and/or aquatic animals. More in particular,
the mammal is at least one livestock and/or animal cell line;
the bird is at least one poultry; and/or
the aquatic animal is at least one crustacean, cephalopod and/or fish.

Livestock may be rearing animals selected from livestock or poultry. In particular, livestock may include cattle, sheep, pigs, goats, horses, camels, donkeys, mules, rabbits and the like and poultry may include chickens, turkeys and other gallinaceous birds, ducks, geese, quail, and the like. As used herein, the term ‘livestock’ may also include poultry and refer to any farm animal or animal that may be used in agriculture.

As used herein, the term “aquatic animal” refers to any organism that lives entirely in water or that lives predominantly in water, especially compared with terrestrial animals. In particular, the aquatic animal according to any aspect of the present invention may be any animal in the animal kingdom that lives predominantly in water. These aquatic animals may live in different water forms, such as seas, oceans, rivers, lakes, ponds, etc. More in particular, the aquatic animal according to any aspect of the present invention may be may any fish, cephalopod, aquatic molluscs, or aquatic crustaceans, at all life stages, including eggs, sperm and gametes. Even more in particular, the ‘aquatic animal’ means animals of the following species: (i) fish belonging to the superclass Agnatha and to the classes Chondrichthyes, Sarcopterygii and Actinopterygii; (ii) aquatic molluscs belonging to the phylum Mollusca; and (iii) aquatic crustaceans belonging to the subphylum Crustacea. Even more in particular, the aquatic animal according to any aspect of the present invention may be aquatic animals used in aquaculture. Some non-limiting examples of aquatic animals according to any aspect of the present invention include barramundi, carp, catfish, halibut, marbled crayfish, marine and brackish fishes, marine shrimp, mitten crabs, mussels, oysters, pangasius, rainbow trout, salmonids, scallops, sea bass, sea bream, soft-shelled crabs, soft-shelled turtles, tiger prawns, tilapia, turbot, white-leg prawn, shrimp, octopus, squid and other decapod crustaceans, bivalves and gastropods.

The animal cell line may be immortal Chinese Hamster Ovary cell line (CHO) derived from Cricetulus griseus, Vero cell line isolated from kidney epithelial cells extracted from an African green monkey (i.e., Chlorocebus sp.). HeLa cell line immortalized from human beings and the like.

In one example, the mammal may be a human being and the array may comprise CpG sites that are directed to different parts of the human being, for example CpG sites related to the human skin.

In particular, the array according to any aspect of the present invention may comprise a first and second animal species selected from the group consisting of salmon, shrimp, swine, chicken, crayfish, CHO, and at least one virus.

The respective CpG sites for each species was categorically selected. In particular, the CpG sites for individual species were specifically selected based on their methylation values observed from earlier experimental data. This includes environmental specific CpG sites which may also be referred to as dynamic sites; CpG sites from dynamic regions such as Low methylated Regions (LMRs) and Differentially Methylated Regions (DMRs) and CpG sites from regulatory regions of candidate genes from pathways which are significant in certain biological context. Examples of biological contexts that might affect CpG site methylation include exposure of animals to antibiotic treatment compared to antibiotic free animals, animals experiencing inflammation compared to healthy controls and animals reared in different geographical regions. Almost all highly valuable CpG targets for both mammals and invertebrates have been represented on the array according to any aspect of the present invention using this design strategy. In particular, the CpG targets on the array according to any aspect of the present invention are not conserved among all species represented and/or have different purposes in different species presented.

“Environmental specific CpG sites” refer to CpG sites which are differentially methylated in response to environmental conditions or exposures. For example, CpG site methylation can be affected by environmental forces like diet, stress, drugs, or pollution among many others. As disclosed in WO2022023208, environment-specific “epigenetic fingerprints” on genomes of various animals have been found and these environmental specific CpG sites may be used to identify the geographic origin of these animals. Accordingly, the term “Environmental specific CpG sites” as used herein refers to CpG sites of an animal genome, and these may greatly vary depending on the taxon or species of animal, that may be used to distinguish one geographic location from another based on one or more environmental parameters. These environment specific CpG sites may also be referred to as dynamic CpG sites. The methylation status of these environmental specific CpG sites may thus vary depending on changes in the environmental or specifically environmental parameters. Such environmental parameters depend on the habitat of the animal and may be different in case the animal is cultured in water, or is grown in soil, or may be selected from a food or air parameter etc. For example, for sweet water crabs (such as the marbled crayfish), environmental parameters may be selected from pH, water hardness, manganese content, iron content, and aluminum content. In another example, a habitat for an animal that lives in water, can be selected from standing or flowing waters such as lakes, rivers, aqua farms, other pools or bodies of water or ponds. A geographic origin shall be understood to be the geographic location that is considered to be a habitat wherein the animal was spawned and/or cultured, or at least cultured for a significant time during their lifetime. Accordingly, ‘Environmental specific CpG sites’ refer to CpG sites, namely dynamic CpG sites where the methylation is changed based on varying environmental parameters.

Low Methylated Region (LMR) is a region of the genome wherein less than 60% of CpGs in that region are methylated. More in particular, less than 50%, 40%, 30%, 20% or 10% of the CpGs in the LMRs are methylated. Any method known in the art may be used to identify or detect LMRs in the genomic DNA. Well known methods include using programmes such as MethylSeekR. In particular, LMRs in the genomic DNA have at least three consecutive CpGs and have no single nucleotide polymorphisms (SNPs) in any of the CpG positions. Even more in particular, LMRs in the genomic DNA are identified based on the method disclosed at least in Burger, L., (2013) Nucleic Acids Research, 41 (16): e155 and/or Stadler, M., (2011) Nature 480, 490-495. LMRs are known to have an average methylation ranging from 10% to 50%; are regions of low CG density which do not overlap with CpG islands; tend to be enriched for H3K4me1, DHSs, and p300/CBP; and/or are primarily located distal to promoters in intergenic or intronic regions. In particular, LMRs:
have an average methylation ranging from 10% to 50%,
are regions of low CG density;
are enriched for Histone H3 monomethylated at lysine 4 (H3K4me1), DNase I hypersensitive sites (DHSs) and transcriptional coactivators CREB binding protein (CPB) and p300;
are primarily located distal to promoters in intergenic or intronic regions; and/or
have no single nucleotide polymorphisms (SNPs) in any of the CpG positions.

Low-methylated regions (LMRs) represent a key feature of the dynamic methylome. LMRs are local reductions in the DNA methylation landscape and represent CpG-poor distal regulatory regions that often reflect the binding of transcription factors and other DNA-binding proteins. LMRs were originally described in the mouse (Stadler et al. (2011) Nature: 480, 490-95). Evolutionary conservation of LMRs beyond mammals has remained unexplored.

Differentially methylated regions (DMRs) are genomic regions with different methylation statuses among multiple biological samples like tissues, cells, individuals, etc. These are genomic regions that differ between phenotypes. The statistical power is likely to be greater when adjacent DMPs are considered together as a whole [Gu H et al (2010) Nat Methods 2010; 7:133-6]. The lengths of the DMRs may range between a few hundred to a few thousand bases [Rakyan et al (2011) Nat Rev Genet 12:529-41, 2011, Bock C (2012) Nat Rev Genet 2012; 13:705-19].

DMRs may occur throughout the genome but have been identified particularly around the promoter regions of genes, within the body of genes, and at intergenic regulatory regions. There are two types of regions, predefined or user defined. Regions with special biological meaning, such as CpG islands, CpG shores, UTRs and so on, are predefined. Many traditional statistical testings, including t-test and Wilcoxon rank sum test, can be performed at a region level. For user-defined regions, criteria such as a fixed region length, fixed numbers of significant and adjacent CpG sites, significant and smoothed estimated effect sizes, etc.

The array according to any aspect of the present invention further comprises:
at least one probe molecule specific for at least one single nucleotide polymorphism (SNP) of the first species of animal; and
at least one probe molecule specific for at least one SNP of the second species of animal.

These probes specific for SNPs may be used for SNP genotyping, which is the measurement of genetic variations of SNPs between members of a species. In particular, an SNP is a single base pair mutation at a specific locus, usually consisting of two alleles (where the rare allele frequency is >1%) that are conserved during evolution. These probes enable the identification of a species, particularly breed of a species. In particular, when a DNA sample is introduced to the array according to any aspect of the present invention, these probes specific to SNPs can be used to determine if the sample is from the first and/or second species of animal found on the array and whether there is DNA from another species other than the first and second animal species that has contaminated the DNA sample.

The array according to any aspect of the present invention comprises at least a third plurality of distinct locations, each location having at least one probe molecule comprising a nucleic acid sequence complementary to a CpG site from a third plurality of CpG sites of a third animal species. In this example, the three animal species may be chicken, crayfish and CHO cells.

The first animal species may be chicken and the plurality of CpG sites comprises at least:
dynamic CpG sites, CpG sites of promoters, and/or CpG sites in Low Methylated Regions (LMRs).

The term ‘CpG sites of promoters’ herein refers to any CpG site that may be found on gene promoter regions in for example immune system genes (IRF5, STAT3, TBK1, SOCS1), feed-linked genes (RAC2, VCAM1, CTSS and TLR4), antibiotics linked genes (RRAD, PDK4, SGK1, PTPRT), breast muscle development genes (Myopathy) (ARNT2, EYA2, PTGS1, CADM1) and the like. As used herein, the terms “promoter” or “gene promoter” used interchangeably with the terms ‘regulatory region’ or ‘regulatory sequence’ refers to the respective contiguous gene DNA sequence extending from 1.5 kb upstream to 1.5 kb downstream relative to the transcription start site (TSS), or contiguous portions thereof. In particular, ‘regulatory region’ refers to the respective contiguous gene DNA sequence extending from 1.5 kb upstream to 0.5 kb downstream relative to the TSS. In some examples, ‘regulatory region’ refers to the respective contiguous gene DNA sequence extending from 1.5 kb upstream to the downstream edge of a CpG island that overlaps with the region from 1.5 kb upstream to 1.5 kb downstream from TSS (and in such cases, may thus extend even further beyond 1.5 kb downstream), and contiguous portions thereof.

The second animal species may be crayfish and the plurality of CpG sites comprises at least: 1 dynamic CpG sites, CpG sites in methylated repeats in the crayfish genome, and/or CpG sites in immune system linked genes, meiosis genes, and DNMT1; and/or
The term ‘CpG sites in dynamic repeats in the crayfish genome’ used herein refers to the CpG sites in transposable elements that showed differential methylation between Procambarus virginalis and Procambarus fallax.

The other CpG sites of interest from the crayfish include CpG sites in immune system linked genes, meiosis genes, and DNA methyltransferase 1 (DNMT1).

‘Immune system linked genes’ in the crayfish include genes such as HSPB1, CL17A, ARSH, SPB9, AGO2 and the like.

‘Meiosis genes’ in relation to crayfish include genes such as POLO-G, CDK10, RECQ4, and RAD54. ‘CpG sites in DNA methyltransferase 1 (DNMT1)’ used herein refers to the CpG sites that were differentially methylated between control and DNMT1 knock-out animals.

The third animal species is a CHO cell and the plurality of CpG sites comprises at least:
dynamic CpG sites, and CpG sites found in promoters of metabolic linked genes, protein production linked genes, cell growth and division linked genes, epigenetic linked genes, and viral promoters.

‘Environmental specific or dynamic CpG sites’ in the context of CHO cells refer to the CpG sites that are differentially methylated among different CHO cell lines. The cell lines that were used in this analysis include CHO-K1 (ATCC), CHO-DG44 (Thermo Fisher Scientific), CHO-DXB11 (ATCC), ExpiCHO-S™ cells (Thermo Fisher Scientific), FreeStyle™ CHO-S™ cells (Thermo Fisher Scientific), CHO 1-15 [subscript 500] (ATCC) and Agarabi CHO (ATCC).

‘Metabolic linked genes’ in the context of CHO cells herein refer to genes that are related to several metabolism pathways such as Glycolysis, TCA cycle, Pentose Phosphate pathway, Malate-aspartate shuttle, Amino acid metabolism, Lactate metabolism, Cholesterol biosynthesis,

Nucleotide biosynthesis, Nucleotide sugar biosynthesis etc. A few examples of such genes include Hk2, Pgk1, Idh3a, Pgm1, and Pdha1. A skilled person would easily determine the genes that are found in CHO cells that fall within this category.

‘Protein production linked genes’ used in the context of CHO cells herein refer to genes that are related to cellular processes such as DNA replication and repair, mRNA transcription, mRNA translation, post-translational modifications, and protein folding and export. A few examples of such genes include Gatb, Sec61a2, Ube2e3, Exosc1, Dna2, Pold1 and the like. A skilled person would easily be able to determine the other genes that are found in CHO cells that fall within this category.

‘Cell growth and division linked genes’ used in the context of CHO cells herein refer to genes that are related to cellular processes such as cell cycle regulation, Cytoskeleton-related elements, cell signalling, nucleotide metabolism, and cell death. A few examples of such genes include Camk1, Cd82, Cdk4, Col1a1, and Ctsb. Again, a skilled person would easily be able to determine the other genes that are found in CHO cells that fall within this category.

‘Epigenetic linked genes’ used in the context of CHO cells herein refer to genes that are related to epigenetic modifications such as DNA methylation pathway, DNA demethylation pathway, Folate and Methionine cycle, and Histone modifications. A few examples of such genes include Hat1, Shmt1, Bhmt, Dnmt1, and Ehmt1. A skilled person would easily be able to determine the other genes that are found in CHO cells that fall within this category.

The term ‘Viral promoters’ used in the context of CHO cells herein refer to promoter and enhancer of at least the cytomegalovirus (CMV) and simian vacuolating virus 40 (SV40).

The array according to any aspect of the present invention may comprise at least 4, 5, 6, 7, 8, 9, 10 and even more plurality of distinct locations, limited by the size of the array.

In one example, the array according to any aspect of the present invention may comprise at least three pluralities of distinct locations, each plurality of distinct locations specific to the CpG sites of an animal species and where the three animal species may be chicken, crayfish and CHO cells.

In this example, when the first animal species is chicken, the plurality of CpG sites comprises at least:
about 60, 55, 50, 45, 40, 35, 30, 25, 20, 15% dynamic CpG sites or 15-60, 15-55, 15-50, 15-45, 15-40, 15-35, 15-30, 15-25, 15-20, 20-60. 20-55, 20-50, 20-45, 20-40, 20-35, 20-30, 20-25, 25-60, 25-55, 25-50, 25-45, 25-40, 25-35, 25-30, 30-60, 30-55, 30-50, 35-60, 35-55, 35-50, 35-45, 35-40, 40-60, 40-55, 40-50, 40-45, 45-60, 45-55, 45-50% dynamic CpG sites;
about 80, 75, 70, 65, 60, 55, 50, 45, 40, 35, 30, 25, 20% CpG sites of promoters and/or genes or 30-80, 30-75, 30-70, 30-65, 30-60, 30-55, 30-50, 30-45, 30-40, 35-80, 35-75, 35-70, 35-65, 35-60, 35-55, 35-50, 35-45, 35-40, 40-80, 40-75, 40-70, 40-65, 40-60, 40-55, 40-50, 40-45, 45-80, 45-75, 45-70, 45-65, 45-60, 45-55, 45-50, 50-80, 50-75, 50-70, 50-65, 50-60, 50-55, 55-80, 55-75, 55-70, 55-65, 55-60, 60-80, 60-75, 60-70, 60-65, 65-80, 65-75, 65-70 70-80, 70-75% CpG sites of promoters and/or genes; and/or
about 30, 25, 20, 15, 10, 5% CpG sites in LMRs or 5-30, 5-25, 5-20, 5-15, 5-10, 10-30, 10-25, 10-20, 10-15% CpG sites in LMRs.

In particular, the first animal species in the array according to any aspect of the present invention is chicken and the plurality of CpG sites comprises at least:
about 52% dynamic CpG sites,
about 36% CpG sites of promoters, and/or
about 12% CpG sites in Low Methylated Regions (LMRs)

In this example, when the second animal species is crayfish, the plurality of CpG sites comprises at least:
about 90, 85, 80, 75, 70, 65, 60, 55, 50, 45, 40, 35, 30% dynamic CpG sites or 30-90, 30-85, 30-80, 30-75, 30-70, 30-65, 30-60, 30-55, 30-50, 30-45, 30-40, 35-90, 35-85, 35-80, 35-75, 35-70, 35-65, 35-60, 35-55, 35-50, 35-45, 35-40, 40-90, 40-85, 40-80, 40-75, 40-70, 40-65, 40-60, 40-55, 40-50, 40-45, 45-90, 45-85, 45-80, 45-75, 45-70, 45-65, 45-60, 45-55, 45-50, 50-90, 50-85, 50-80, 50-75, 50-70, 50-65, 50-60, 50-55, 55-80, 55-75, 55-70, 55-65, 55-60, 60-90, 60-85, 60-80, 60-75, 60-70, 60-65, 65-80, 65-75, 65-70 70-80, 70-75% dynamic CpG sites;
about 50, 45, 40, 35, 30, 25, 20, 15, 10, 5, 4, 3, 2, 1% CpG sites found in methylated repeats in the crayfish genome or 5-50, 5-45, 5-40, 5-35, 5-30, 5-25, 5-20, 5-15, 5-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 35-45, 35-40, 40-50, 40-45, 45-50% CpG sites found in methylated repeats in the crayfish genome;
about 30, 25, 20, 15, 10, 5% CpG sites in immune system linked genes, meiosis genes, and DNMT1 or 5-30, 5-25, 5-20, 5-15, 5-10, 10-30, 10-25, 10-20, 10-15% CpG sites in immune system linked genes, meiosis genes, and DNMT1. In particular about 1, 2, 3, 4, 5% CpG sites are associated with the DNMT1 gene and about 15, 14, 13, 12, 11, 10, 9, 8, 7, 6, 5% of the CpG sites are associated with immune system linked genes and/or meiosis genes.

In particular, the second animal species in the array according to any aspect of the present invention is crayfish and the plurality of CpG sites comprises at least:
about 83% dynamic CpG sites,
10-about 5% CpG sites found in methylated repeats in the crayfish genome, and/or
about 10% CpG sites in immune system linked genes, meiosis genes, and/or
about 3% CpG sites in DNMT1.

In this example, when the third animal species is a CHO cell, the plurality of CpG sites comprises at least:
about 50, 45, 40, 35, 30, 25, 20, 15, 10, 5% dynamic CpG sites or 5-50, 5-45, 5-40, 5-35, 5-30, 5-25, 5-20, 5-15, 5-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 35-45, 35-40, 40-50, 40-45, 45-50% dynamic CpG sites; and
about 95, 90, 85, 80, 75, 70, 65, 60, 55, 50% CpG sites found in promoters, metabolic linked genes, protein production linked genes, cell growth/division linked genes, methylation linked genes, and viral promoters or 50-95, 50-90, 50-85, 50-80, 50-75, 50-70, 50-65, 50-60, 50-55, 55-95, 55-90, 55-85, 55-80, 55-75, 55-70, 55-65, 55-60, 60-95, 60-90, 60-85, 60-80, 60-75, 60-70, 60-65, 65-95, 65-90, 65-85, 60-80, 65-75, 65-70, 70-95, 70-90, 70-85, 70-80, 70-75, 65-95, 65-90, 65-85, 65-80, 65-75, 65-70, 65-65, 65-60, 25 60-95, 60-90, 60-85, 60-80, 60-75, 60-70, 60-65, 65-95, 65-90, 65-85, 65-80, 65-75, 65-70, 70-95, 70-90, 70-85, 70-80, 70-75, 75-95, 75-90, 75-85, 75-80, 80-95, 80-90, 80-85, 85-95, 85-90, 90-95 CpG sites found in promoters, metabolic linked genes, protein production linked genes, cell growth/division linked genes, methylation linked genes, and viral promoters. 30

In particular, the third animal species in the array according to any aspect of the present invention is a CHO cell and the plurality of CpG sites comprises at least:
about 28% dynamic CpG sites, and/or
about 72% CpG sites found in promoters, metabolic linked genes, protein production linked genes, cell growth/division linked genes, methylation linked genes, and viral promoters. In particular, about 63% of the CpG sites are found in promoters, metabolic linked genes, protein production linked genes, cell growth/division linked genes, and methylation linked genes, and about 9% of the CpG sites are found in viral promoters.

In one example, the array according to any aspect of the present invention may comprise at least 3 animal species, wherein the first animal species may be chicken and about 45-50% of the total number of CpG sites on the array are from chicken, the second animal species may be crayfish and about 10-20% of the total number of CpG sites on the array are from crayfish, and the third animal species may be CHO cell lines and about 30-40% of the total number of CpG sites on the array are from CHO cell lines.

In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific deviation for a numerical value for a given technical effect will depend on the nature of the technical effect. For example, a natural or biological technical effect may generally have a larger such deviation than one for a man-made or engineering technical effect.

According to another aspect of the present invention, there is provided use of the array according to any aspect of the present invention for predicting the biological age of a test animal.

The term “chronological age” refers to the calendar time that has passed from birth/hatch.

The biological age depends on the biological state or condition of an individual or of a population and takes into account the circumstances of life (such as stress, nutrition, etc.). The terms “epigenetic age”, “methylation age”, and “biological age” have identical meanings and are used interchangeably in the context of the present application.

The term “test” used in conjunction with the term animal herein refers to an animal that is introduced to the array according to any aspect of the present invention and is the basis for an analysis application of the present invention. A ‘test animal’ is therefore an animal being tested according to any aspect of the present invention or a profile being obtained or generated in this context. Conversely, the term “reference” or ‘control’ shall denote, mostly predetermined, entities which are used for a comparison with the test entity. In particular, a ‘test animal’ refers to an animal being tested to determine any feature of the animal (i.e. biological age, geographical origin, rearing method etc.) where the methylation status has to be determined and a ‘control’ refers to an animal where the features as mentioned above are already known and where the methylation status is already known and used as a reference.

According to a further aspect of the present invention, there is provided a use of the array according to any aspect of the present invention for determining if a test animal and/or a test animal from which a product is derived has been treated and/or is currently undergoing treatment with at least one antibiotic and/or veterinary chemical.

As used herein the term ‘antibiotic’ refers to any medicine that may be fed to the terrestrial animal for therapeutic and/or preventive purposes. The antibiotic may be administered by any method known in the art. The antibiotic may be fed orally to the aquatic animal according to any aspect of the present invention in the animal feed, or water where the aquatic animal is farmed such that it is ingested or used as a bath for external body infections. In another example, the antibiotic may be injected into the aquatic animal. A skilled person would understand the best way to provide the antibiotic to the animal based on the specific biological taxon of the animal, the type of antibiotic and the disease to be treated or prevented. In particular, the antibiotic according to any aspect of the present invention may be selected from the group of classes consisting of amphenicols, aminocyclitols, aminoglycosides, ansamycins, beta-lactams, carbaephem, carbapenems, cephalosporins, chloramphenicol, fluoroquinolones, glycopeptides, glycylcyclines, ketolides, lincosamides, lipopeptides, macrolides, monobactams, nitrofurans, nitroimidazoles, oxazolidinones, penicillins, phosphonic acid derivatives, pleuromutilins, polymyxins, polypeptides, quinolones, rifamycins, riminofenazines, steroid antibacterials, streptogramins, sulfonamides, tetracyclines, and trimethoprim. More in particular, the antibiotic may be selected from the group consisting of tetracycline and fluoroquinolones, particularly norfloxacin.

The test animal according to any aspect of the present invention may be fed with at least one or more antibiotics mentioned above simultaneously or consecutively. The contact of antibiotics with the test animal may bring about epigenetic changes, at least DNA methylation changes, that may then be determined using the method according to any aspect of the present invention. The concentration of antibiotics in each dose and/or the period of time the antibiotic has been given to the test animal may affect the extend of differential methylation in the test animal relative to the control animal. It is within the knowledge of a skilled person to determine the concentration of each dose and the period of antibiotic exposure that the test animal requires depending on whether the antibiotic is given for preventive or therapeutic measures.

As used herein the term ‘veterinary chemical’ refers to drugs or medicines used to treat or prevent disease, injury and pests in animals. In particular, ‘veterinary chemical’ may refer to an anti-parasitic, an anti-viral, a feed additive, a water additive, a disinfectant, glutaraldehyde, formalin, mixtures thereof and the like. The veterinary chemical may be administered by any method known in the art to the test animal.

The test animal used in the method according to any aspect of the present invention may be brought into contact with both an antibiotic and a veterinary chemical simultaneously and/or consequently. The change in the internal environment of the test animal leads to an epigenetic change and this can be determined using the array according to any aspect of the present invention.

The array according to any aspect of the present invention may also be used to determine if a test animal and/or a test animal from which a product is derived has been treated and/or is currently undergoing treatment with at least one antibiotic, and if so, determining the distinct class of antibiotics with which the test animal is being treated and/or is currently undergoing treatment.

According to yet another aspect of the present invention there is provided a use of the array to determine if a test animal and/or a test animal from which a product is derived has been treated and/or is currently undergoing treatment with at least one antibiotic, and if so, determining if the antibiotic is used as a growth promotant or as a therapeutant.

As used herein the term ‘growth promotant’ refers to the antibiotic being used to help increase the efficiency of animal production by increasing weight gain and product output. The antibiotic may be used as a growth promotant in contrast to it being used as a therapeutant (i.e. for treatment of a disease).

As used herein, the term ‘animal-derived product’ refers to products that originate from animals. In particular, the term ‘test animal-derived product’ refers to the sample or subject in question that is to be introduced to the array according to any aspect of the present invention. These products from animals may include meat and meat products, also including fat, flesh, blood, processed meat, and lesser-known products, such as isinglass and rennet, poultry products (meat and eggs), dairy products (milk and cheese), and non-food products such as fibre (wool, mohair, cashmere, leather, and the like). Animal-derived products may also include products that can be made using animal products (e.g., fat) such as soap, creams, and such. In one example, the animal-derived product is meat, eggs, blood, brain, sperm, milk and any other tissue or sample that provides genomic DNA. In particular, the animal-derived product is meat. In one example, the animal-derived product sample may be a single type of meat, different types of meat, a single part of a type of meat, different parts of a single type of meat or different parts of different types of meat. In the event the animal is an aquatic animal, these products from animals may include meat and meat products, also including eggs, fat, flesh, blood, processed meat and lesser-known products, and non-food products such as fibre (shells, scales and the like). Animal-derived products may also include products that can be made using animal products (e.g. fish oil) such as tablets, powder and such. In one example, the animal-derived product is meat, eggs, blood, brain, shell, scale, skin, tissue, abdominal muscle tissue or any other tissue or sample that provides genomic DNA. In particular, the animal-derived product is meat, skin, blood, trimmings or any organ from the aquatic animal. In particular, trimmings are used as biproducts for fish meal/oil which end up in the animal feed industry or pets. The sample may be from any biological entity having a DNA genome and DNA genome methylation. In particular, the methylation site is a CpG site.

According to yet further aspect of the present invention, there is provided a use of the array according to any aspect of the present invention to determine if a test animal from which a product is derived underwent a withdrawal period of no treatment with at least one antibiotic and/or veterinary chemical prior to the product being obtained.

As used herein, the term ‘withdrawal period’ refers to the period from the time point where the animal is no longer fed the antibiotic and/or veterinary chemical to the point where the remaining antibiotic is broken down in the body until it becomes a non-functional agent and is finally, eliminated from the body of the animal. Withdrawal periods of different antibiotics may vary from 1 or 2 days to couple of weeks. A “withdrawal” period is required from the time antibiotics are administered until it is legal to slaughter or kill the animal or to derive products from the animal. The time it therefore takes the body to break down the antibiotic until it is no longer functional, or present is called the withdrawal time (or withdrawal period). Once the withdrawal period has passed the antibiotic has been eliminated from the animal's system.

According to yet a further aspect of the present invention, there is provided a use of the array according to any aspect of the present invention for determining a distinct certification of a test animal-derived product sample. In particular, the distinct certification of the animal derived product sample is whether the animal has been slaughtered by a single cut across the throat severing both carotid arteries, both jugular veins, both vagus nerves, the trachea and the esophagus and/or of the test animal having been bled to death.

The term ‘certification of quality’ refers to a certificate or a confirmation given by designated certification agencies that endorse the quality of a particular animal derived product, including food for use and/or consumption by human beings. The term ‘certification of quality’ is used interchangeably with the term ‘certification’. These certifications are usually found on the packaging of the animal-derived product including food to be consumed and are printed by the manufactures of the products. Examples of certifications of distinct food quality may include ‘Haltungsform’, ‘Tierwohl’, ‘Ohne Gentechnik’, ‘halal’, ‘kosher’, and other safe labels that confirm that a product sold has been prepared in accordance with specific religious or safety regulations. Specifically, the term ‘certification of food quality’ refers to a certificate or a confirmation given by designated certification agencies that endorse the quality, source or means of slaughter of a particular food for consumption by human beings. According to any aspect of the present invention, the certified quality may be a distinct certified food quality, or distinct certification and this may be kosher, non-kosher, halal or non-halal.

In one example, the distinct certified food quality or certification of the sample X according to any aspect of the present invention may be kosher, non-kosher, halal or non-halal. More in particular, kosher or halal refers to the sample X originating from an animal that was slaughtered by a single cut across the throat severing both carotid arteries, both jugular veins, both vagus nerves, the trachea and/or the esophagus. Even more in particular, the animal is drained of blood.

The term ‘kosher’ used in combination with food according to any aspect of the present invention refers to food that conforms to Jewish dietary regulations of kashrut (dietary law) or food that may be consumed according to halakha (law). Kosher used in relation to meat relates particularly to a manner in which animals are prepared for consumption. According to Jewish tradition, meat may be considered kosher when the meat comes from animals that have been slaughtered according to Jewish law where the animal is killed by a single cut across the throat to a precise depth, severing both carotid arteries, both jugular veins, both vagus nerves, the trachea and the esophagus, no higher than the epiglottis and no lower than where cilia begin inside the trachea, causing the animal to bleed to death. Such slaughter is to be carried out using a large, razor-sharp knife, which is checked before each slaughter to ensure that it has no irregularities (such as nicks and dents). The slaughter is usually also carried out by a shochet or a rabbi. Kosher meat usually refers to most meats excluding pig. In particular, kosher meat may be selected from beef, chicken, lamb, mutton, goat meat and mixtures thereof. Kosher meat does not include shellfish, which under Jewish tradition is not permitted for consumption. Although Jewish traditions permit consumption of vertebrate fish, since there is no special method of slaughtering vertebrate fish, all vertebrate fish may be considered kosher. Any food or meat that does not fall within the definition of ‘kosher’ will then be considered as ‘non-kosher’.

The term ‘halal’ used in combination with food according to any aspect of the present invention refers to food that conforms to Islamic dietary laws and especially meat processed and prepared in accordance with those requirements. Similar to the way kosher meat is prepared, in Islamic tradition, animals are slaughtered according to Dhabihah where the animal is slaughtered using a cut across the neck with a non-serrated sharp blade in a single clean attempt to make an incision that cuts the front of the throat, oesophagus and jugular veins but not the spinal cord. In addition to the direction, permitted animals should be slaughtered upon utterance of the Islamic prayer Bismillah. The animal must also be drained of blood after the slaughter. The slaughter must be performed by an adult Muslim. Halal meat usually refers to most meats excluding pig. In particular, halal meat may be selected from beef, chicken, lamb, mutton, goat meat and mixtures thereof. Although Islamic traditions permit consumption of shellfish and vertebrate fish, since there is no special method of preparing shellfish, all shellfish and vertebrate fish may be considered halal. Any food or meat that does not fall within the definition of ‘halal’ will then be considered as ‘non-halal. The definition of halal is further provided in https://www.smiic.org/en/project/24 (Organisation of Islamic Cooperation (OIC)/Standards and Metrology Institute for the Islamic Countries (SMIIC), OIC/SMIIC 1:2019 General Requirements for Halal Food. accessed on 8 Jun. 2022).

In one example, the distinct certification of the animal derived product sample according to any aspect of the present invention is based on a type of animal husbandry that the test animal was reared under.

In one example, the distinct certification or certification of sample X may be based on a type of animal husbandry that the test animal was reared under. In Germany, this is labelled as ‘Haltungsform’. There are at least four types/conditions under which the animals may be reared. These four levels of animal husbandry include Stable housing (Stallhaltung), Stable housing Plus (StallhaltungPlus), Outside climate (Außenklima) and Premium (Premium), these are also known as Haltungsform 1, 2, 3 and 4 respectively. Animal products derived from animals bred under different animal husbandry conditions may result in a different DNA methylation profile. The distinct type of animal husbandry may vary depending on the country where the method according to any aspect of the present invention is carried out. Regardless of different terminology used in different countries to describe different distinct animal husbandry practices, the overall concept of the method according to any aspect of the present invention is the same and applicable in any one of these countries.

For example, in Germany, the different distinct types of animal husbandry techniques practiced on livestock and poultry may be labelled ‘Haltungsform’ and as mentioned above, are officially and accepted by the industry to be divided into least four types/conditions under which the animals may be reared. These four levels of animal husbandry include Stable housing (Stallhaltung), Stable housing Plus (StallhaltungPlus), Outside climate (Außenklima) and Premium (Premium). Similarly, in France the livestock and poultry may be labelled ‘label rouge’, ‘organic’, or with other pictograms that display the farming methods the animal went through before the animal derived product was obtained. In the United Kingdom livestock and poultry the Red Tractor Food Assurance certification scheme exists which includes at least three levels of animal husbandry including Certified Standards, Enhanced Welfare and Free Range. Other labels existing in the United Kingdom include RSPCA Assured which certify specific animal welfare standards and several organic meat certifying schemes such as the Organic Farmers and Growers Certification and the Soil Association Organic Standard. Examples of meat certification in the United States of America (USA) includes those provided by the United States Department of Agriculture (USDA), which include Grade A Carcass Quality and Organic certifications as examples. The USDA also approves some third-party certification schemes such as provided by the nonprofit A Greener World, which include Certified Animal Welfare Approved defining husbandry related to animal welfare and Certified Grassfed defining specific feed types in animal husbandry.

According to yet another aspect of the present invention, there is provided a use of the array according to any aspect of the present invention for identification of the geographic origin of a test animal-derived product.

The term “geographic origin” used herein relates to a geographic location which is distinguished from other geographic locations by one or more environmental parameters of the test animal. Such environmental parameters depend on the habitat of the animal and may be different in case the animal lives or is cultured in water, on or in soil, or may be selected from a food or air parameter etc. In one example, for sweet water crabs (such as the marbled crayfish), relevant environmental parameters may be selected from pH, water hardness, manganese content, iron content, and aluminum content. However, environmental parameters that are relevant may vary greatly depending on the taxon or species of the animal. Similarly, a habitat for an animal that lives in water may also vary for example, these habitats can be selected from standing or flowing waters such as lakes, rivers, aqua farms, other pools or bodies of water or ponds. A geographic origin shall be understood to be a geographic location that is considered to be the habitat, where the test animal, was birthed, hatched and/or reared, or at least reared for a significant time during their lifetime.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

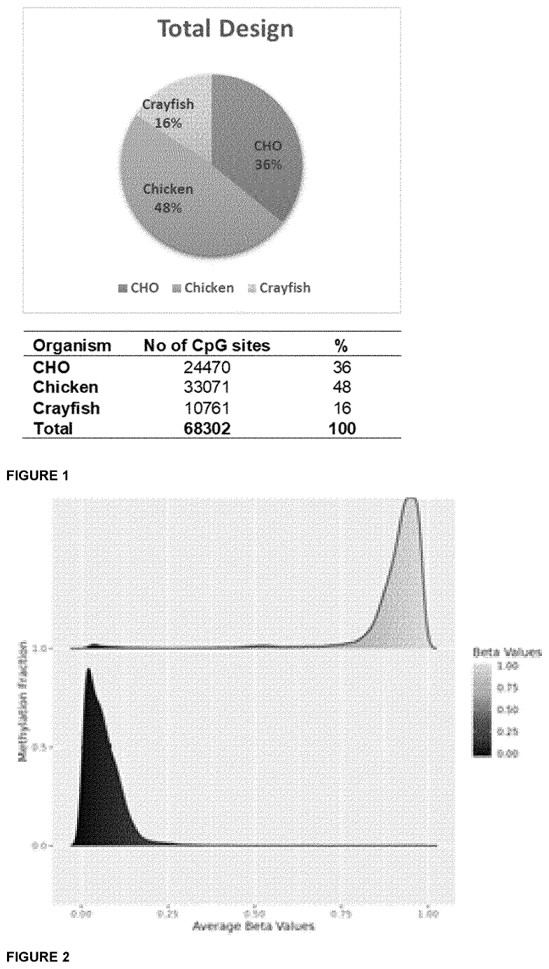

A customizable methylation bead BeadChip array (Illumina) was designed for functionality with three distinct animal species: Chinese Hamster Ovary cell lines (CHO), chicken and crayfish (FIG. 1).

The end design contained nearly 70,000 CpG sites. For designing the array, the candidate CpG sites for each species was identified using the following methods for each category (Table 1):

TABLE 1
Breakdown of CpG site categories per species in
the multispecies methylation bead-array design.

| | | No of | |
|---|---|---|---|
| Species | CpG Site Category | CpG Sites | % |
| Chicken | Environmental specific/ dynamic probes | 17289 | 52% |
| | Gene | 11956 | 36% |
| | LMR | 3883 | 12% |
| | Total Chicken CpG Sites | 33071 | 100% |
| Chinese | Environmental specific/ dynamic probes | 6872 | 28% |
| Hamster | gene | 15300 | 63% |
| Ovary | viral | 2298 | 9% |
| | Total CHO CpG Sites | 24470 | 100% |
| Crayfish | Environment specific/ dynamic VMRs | 8906 | 83% |
| | DNMT1-responsive | 301 | 3% |
| | Dynamic repeats | 500 | 5% |
| | Immune/meiosis genes | 1034 | 10% |
| | Total Crayfish CpG sites | 10741 | 100% |

### Environment Specific CpG Sites or Dynamic CpG Sites

Differentially methylated Positions (DMPs) were identified from sequencing data (WGBS/RRBS). Samples from various environmental conditions were collected, the sequencing data was then processed and analyzed to identify the CpG sites contributing to various environmental factors including location, various treatment conditions etc. (Tables 2 and 5)

### LMR/Clock Based CpG Sites

These sites were selected using chicken methylation age clock developed using penalized regression model to regress the chronological age as described in Raddatz, G., Commun Biol 4, 76 (2021). (Table 4).

### Gene Promoter Based CpG Sites

3 CpG sites per gene promoter were identified from all promoters (exploratory) and candidate genes based on the species-specific requirement as follows:
For chicken, probes were designed using unpublished data for promoters (exploratory) and candidate genes with emphasis on immune system genes, feed-linked, genes, antibiotics linked genes, breast muscle development genes e.g. those related to breast meat myopathies (some examples are provided in Table 3).
For CHO, probes were designed for all promoters (exploratory) and candidate genes with emphasis on metabolic linked genes, protein production linked genes, cell growth/division linked genes, methylation linked genes, Viral promoters and enhancers such as CMV and SV40 (some examples are provided in Table 6).
For crayfish, probes were designed for immune system linked genes, meiosis genes, DNMT1 responsive CpG sites (some examples are provided in Table 7).

The CpG sites were annotated with illumina design information provided and the final list of the CpG sites represented on the array were selected based on the designability score, probe type and strand type of each site.

TABLE 2a
Representative CpG sites from the chicken dynamic probes on the multispecies DNA-methylation
bead array chip. Table shows an example 432 of the total 17289 chicken dynamic sites.

| Chrom. 1 | Chrom. 2 | Chrom. 3 | Chrom. 4 | Chrom. 5 | Chrom. 6 | Chrom. 7 | Chrom. 8 | Chrom. 9 | Chrom. 10 | Chrom. 11 | Chrom. 12 |
|---|---|---|---|---|---|---|---|---|---|---|---|
| 81937300 | 348468 | 47955044 | 4396898 | 7177304 | 20558090 | 22252939 | 21208136 | 22375808 | 19246088 | 18993457 | 11520351 |
| 179921173 | 4085817 | 105014227 | 13853754 | 9534985 | 23102332 | 22669337 | 2549725 | 5117318 | 19393142 | 9373410 | 11445914 |
| 120148065 | 560890 | 104937361 | 86427186 | 28371954 | 34155392 | 10588608 | 20849446 | 22098576 | 19172166 | 604551 | 1495387 |
| 583379 | 230608 | 105925476 | 15265798 | 91975 | 23287798 | 12183250 | 4535765 | 5139142 | 1325060 | 19110543 | 9128296 |
| 194818482 | 19761994 | 16404186 | 24224452 | 53937018 | 23101775 | 34963494 | 2300105 | 1203622 | 2366670 | 19446006 | 11620945 |
| 51155986 | 115378315 | 31878747 | 12499792 | 10093568 | 11909490 | 36305441 | 25216849 | 22372745 | 1620591 | 1104215 | 18406931 |
| 180182311 | 32735731 | 96916357 | 9030456 | 23772896 | 23043623 | 27294957 | 21208441 | 23463957 | 2351194 | 18893738 | 1860731 |
| 104736272 | 149487287 | 105642084 | 66645115 | 49728661 | 22260859 | 36220714 | 20863089 | 4895886 | 18703406 | 18796956 | 11590807 |
| 132092314 | 3906142 | 3510279 | 11263934 | 28809936 | 23084284 | 5720167 | 27569306 | 15447031 | 9077361 | 534157 | 6389853 |
| 49249845 | 126258796 | 72911727 | 68799893 | 37213182 | 31713812 | 22278697 | 20406702 | 2570941 | 2549789 | 17931741 | 18274822 |
| 183451291 | 103181599 | 32008051 | 1210412 | 21567169 | 17817163 | 20144015 | 25255677 | 5126097 | 14514552 | 19717943 | 16430242 |
| 15898800 | 517076 | 7681394 | 65734278 | 23114829 | 8009444 | 22355476 | 20527810 | 15923381 | 2278287 | 18845458 | 1005950 |
| 23035111 | 121457012 | 104970025 | 13853782 | 17624727 | 35054349 | 16385498 | 25817267 | 15011351 | 19299872 | 17932900 | 9155473 |
| 187792659 | 61972505 | 4700302 | 1793797 | 16952875 | 2157037 | 15859685 | 4309191 | 4824434 | 6759742 | 609072 | 2458439 |
| 51011667 | 596166 | 4398362 | 13014988 | 21494150 | 10925339 | 22384841 | 20522283 | 22539712 | 20019644 | 1260546 | 1451091 |
| 112396012 | 43340598 | 106824973 | 5301249 | 58203021 | 31713788 | 22355479 | 28653133 | 2563160 | 17581105 | 18993901 | 2462581 |
| 112007740 | 89362067 | 51720329 | 47115579 | 38317337 | 21985159 | 22726341 | 14104042 | 14945101 | 2278255 | 19167220 | 11623915 |
| 22838931 | 35587090 | 16062066 | 90724820 | 37712736 | 34172396 | 12797507 | 20639863 | 2570934 | 1196388 | 18846575 | 2631768 |
| 105349994 | 230450 | 17158827 | 43663533 | 41013895 | 10883262 | 22343953 | 29363057 | 4375270 | 2325485 | 18894011 | 2399498 |
| 4120874 | 244732 | 6568035 | 16627496 | 16908134 | 31713799 | 22544570 | 1515191 | 2570949 | 13143161 | 1768577 | 11581077 |
| 193371147 | 357578 | 55938039 | 31825482 | 54510386 | 23080152 | 13819359 | 5320269 | 15755634 | 19955518 | 19191977 | 11575933 |
| 51175428 | 61680170 | 3017205 | 91259069 | 16894229 | 11461940 | 16352482 | 25357691 | 2570945 | 3475503 | 19015746 | 4932644 |
| 112386033 | 3598760 | 4316013 | 2408484 | 48587507 | 4879826 | 28663811 | 15877567 | 7925956 | 19170486 | 1836233 | 15348276 |
| 50867843 | 386369 | 105653195 | 12334041 | 19190620 | 12306730 | 26948879 | 17636740 | 15052651 | 19913691 | 7976003 | 3216623 |
| 80839115 | 67156322 | 62923341 | 956663 | 117876 | 22128522 | 23692494 | 27675149 | 15386071 | 3437495 | 4306191 | 11620806 |
| 117840268 | 217497 | 67701536 | 15709576 | 42935322 | 20107781 | 36900662 | 20492612 | 4339335 | 20060912 | 4306191 | 6382277 |
| 71193207 | 132687218 | 23381290 | 17938265 | 17060700 | 5252114 | 22511337 | 27552407 | 19601177 | 12896419 | 19061424 | 1879538 |
| 194363008 | 102578170 | 62923411 | 66171221 | 16160754 | 17490625 | 26104573 | 28585144 | 22397224 | 12500816 | 19061424 | 8935754 |
| 194665704 | 57127155 | 9367365 | 1075519 | 11543532 | 9100425 | 12651103 | 5664994 | 15639734 | 6192964 | 7837229 | 1859363 |
| 139082264 | 386444 | 98313656 | 1510080 | 58235927 | 28951531 | 6670399 | 3796288 | 17134839 | 796357 | 19155806 | 11659639 |
| 144511258 | 115215294 | 59271348 | 3855381 | 51776354 | 22192630 | 12703718 | 25249151 | 4195357 | 15860387 | 18796943 | 1780764 |
| 195423585 | 110358918 | 4398377 | 68115499 | 28372136 | 11167235 | 22180715 | 21597804 | 17477648 | 5331017 | 488934 | 9200798 |
| 124174850 | 556054 | 104884071 | 31650242 | 16852665 | 20701036 | 26104560 | 15877559 | 4973594 | 12901038 | 660044 | 8590517 |
| 53679015 | 40618192 | 30383475 | 1119669 | 12176346 | 14574217 | 36218999 | 24338015 | 914779 | 20056828 | 18611325 | 8965899 |
| 53590783 | 117623916 | 30383465 | 51596186 | 397888 | 22521273 | 22740343 | 15127745 | 4459033 | 20056259 | 20211775 | 2263621 |
| 48695447 | 214665 | 105338002 | 84501034 | 26201042 | 22108866 | 16838143 | 9704508 | 7955891 | 12887495 | 1410215 | 6935790 |

TABLE 2b
Representative CpG sites from the chicken dynamic probes on
the multispecies DNA-methylation bead array chip. Table shows
an example 468 of the total 17289 chicken dynamic sites.

| Chrom. 13 | Chrom. 14 | Chrom. 15 | Chrom. 16 | Chrom. 17 | Chrom. 18 | Chrom. 19 |
|---|---|---|---|---|---|---|
| 3224651 | 14141456 | 10860559 | 62976 | 2459797 | 9579784 | 8736228 |
| 3245125 | 4468200 | 1843516 | 62976 | 1065689 | 49394 | 5880980 |
| 15891776 | 12753895 | 7182219 | 215563 | 10127665 | 9820796 | 5919056 |
| 10168394 | 12170050 | 6990774 | 89198 | 956038 | 9889888 | 654262 |
| 2434775 | 4475471 | 1730564 | 67707 | 1057897 | 9879969 | 9527594 |
| 3261395 | 13073114 | 10019593 | 67698 | 2059842 | 10007949 | 176442 |
| 15743093 | 5087250 | 5100142 | 104751 | 1059357 | 9643102 | 1394840 |
| 2432978 | 13762612 | 8443051 | 187766 | 5324534 | 9948577 | 7415487 |
| 10336326 | 8500345 | 9862432 | 225415 | 1005558 | 10518459 | 8672406 |
| 10168366 | 4468141 | 7156376 | 114874 | 9209952 | 8977183 | 7639192 |
| 15907963 | 2654082 | 11109630 | 185338 | 7513891 | 9385662 | 2516377 |
| 3461863 | 14013246 | 7372208 | 89175 | 3825134 | 3836919 | 1988252 |
| 2964456 | 4806286 | 6487320 | 81129 | 9973021 | 9228239 | 676230 |
| 2587372 | 4200583 | 11346422 | 219038 | 8075980 | 9370036 | 5915123 |
| 16858518 | 2122082 | 6132720 | 69153 | 956024 | 2131940 | 6868016 |
| 9205933 | 3363462 | 10027870 | 69109 | 10115271 | 9363995 | 6070675 |
| 859483 | 13647979 | 4674381 | 104763 | 10117779 | 10066580 | 8867087 |
| 10364590 | 14288382 | 6891798 | 193509 | 7755302 | 10413720 | 6066497 |
| 930660 | 14288382 | 10167747 | 163741 | 1131478 | 11027742 | 5381847 |
| 8578113 | 12200256 | 7896188 | 104730 | 10895658 | 9311341 | 5383506 |
| 8424377 | 12717478 | 6227509 | 177073 | 10895664 | 2131935 | 7728180 |
| 1715955 | 6914853 | 7182198 | 225454 | 10895664 | 5105903 | 5919154 |
| 1715955 | 14167458 | 7569372 | 193621 | 8776503 | 9124024 | 5584455 |
| 2591920 | 6978593 | 10917142 | 74668 | 10269319 | 9215765 | 6937917 |
| 3461607 | 13533317 | 7898432 | 111807 | 1864025 | 5539266 | 8762831 |
| 1722467 | 4461743 | 9091433 | 62978 | 8580077 | 6713944 | 5539025 |
| 10092349 | 4155987 | 11264561 | 112007 | 5142579 | 9757765 | 2524036 |
| 13346082 | 13112551 | 9176354 | 112037 | 1123937 | 4470130 | 7620090 |
| 16146762 | 5057704 | 7052290 | 566222 | 10895495 | 9057532 | 1389337 |
| 10457049 | 12762302 | 5615505 | 238088 | 8618707 | 10593085 | 3251550 |
| 1714849 | 928688 | 7099302 | 7465 | 6605830 | 9870834 | 5338399 |
| 1714849 | 2664265 | 6936834 | 17581 | 9781180 | 10373359 | 3255436 |
| 13099011 | 12747865 | 11107678 | 221128 | 1864021 | 11027714 | 319291 |
| 1716207 | 14143362 | 11463092 | 80343 | 9246051 | 9312172 | 4746473 |
| 10191695 | 2406636 | 9446584 | 219737 | 7761230 | 3605186 | 8848114 |
| 10677539 | 2522014 | 10043654 | 218873 | 2058999 | 2262573 | 6872642 |

| Chrom. 20 | Chrom. 21 | Chrom. 22 | Chrom. 23 | Chrom. 24 | Chrom. 25 |
|---|---|---|---|---|---|
| 12008400 | 2627311 | 457240 | 4151444 | 4342967 | 1876695 |
| 12008403 | 6337975 | 2872975 | 5378505 | 5201834 | 2014869 |
| 9848282 | 1214590 | 1434518 | 5365790 | 375808 | 2158533 |
| 9688039 | 4332229 | 2970957 | 5355067 | 4445654 | 27287 |
| 9688100 | 571168 | 1293581 | 4150616 | 5620006 | 2871001 |
| 757415 | 6532483 | 614881 | 4308042 | 5404237 | 1857846 |
| 9711251 | 6554855 | 2878950 | 5242918 | 3863817 | 1886192 |
| 9688000 | 821425 | 2262162 | 2039632 | 27691 | 1385853 |
| 481336 | 4732528 | 2199525 | 2028002 | 5662180 | 2697264 |
| 10794778 | 3937147 | 613063 | 1870015 | 3549396 | 2589999 |
| 9635810 | 4207802 | 2261596 | 1837210 | 4466801 | 2589999 |
| 5255688 | 610423 | 2887292 | 2020261 | 5367885 | 1349368 |
| 10492440 | 6323431 | 2638147 | 4459552 | 2545986 | 1788854 |
| 5369834 | 6526345 | 3543027 | 2239934 | 215454 | 2155125 |
| 13309371 | 6552768 | 545316 | 4956392 | 245523 | 2717815 |
| 7982899 | 3417231 | 2122238 | 2032864 | 364273 | 2387475 |
| 9711248 | 1632794 | 615021 | 4988906 | 259775 | 2697634 |
| 13620507 | 2080396 | 2240079 | 4999716 | 2560987 | 2344212 |
| 11697166 | 3394838 | 1288350 | 5026694 | 2569260 | 1393056 |
| 12008359 | 6301730 | 2775838 | 2098341 | 2404538 | 2671268 |
| 766396 | 741366 | 3549713 | 4145394 | 375697 | 1701110 |
| 502969 | 584413 | 36247 | 1273854 | 5421187 | 269858 |
| 10208627 | 822979 | 4642845 | 5050947 | 356896 | 2519638 |
| 755812 | 5499547 | 1420903 | 4104361 | 289375 | 1617873 |
| 13022291 | 6743890 | 2396019 | 5433201 | 59053 | 13803 |
| 5255634 | 6561529 | 1318567 | 2413944 | 4836258 | 2490698 |
| 10692525 | 668272 | 1293462 | 2339289 | 2002337 | 2631054 |
| 10696133 | 6550064 | 3563999 | 2257738 | 4556685 | 2148992 |
| 629511 | 6549390 | 4636648 | 2106767 | 4535921 | 934056 |
| 629511 | 4245124 | 2413154 | 5390751 | 444726 | 2697472 |
| 8419474 | 541980 | 2439835 | 5360348 | 5075059 | 1438129 |
| 9580751 | 592920 | 451184 | 4220820 | 1770824 | 2702384 |
| 10478805 | 592920 | 2385161 | 5625754 | 1413156 | 1628191 |
| 8419504 | 4869036 | 1322995 | 4151462 | 6208564 | 2400156 |
| 10277213 | 575848 | 2198043 | 4069126 | 1526502 | 1848837 |
| 9936068 | 543520 | 4597487 | 4428828 | 4179180 | 2556214 |

TABLE 2c
Representative CpG sites from the chicken dynamic probes on the multispecies DNA-methylation
bead array chip. Table shows an example 206 of the total 17289 chicken dynamic sites.

| Chrom. 26 | Chrom. 27 | Chrom. 28 | Chrom. 30 | Chrom. 32 | Chrom. 33 | Scaffolds |
|---|---|---|---|---|---|---|
| 2092150 | 4027549 | 950442 | 21104 | 11893 | 1175825 | chr1_NT_456103v1_random 3063 |
| 4370521 | 4160570 | 1718605 | | 34426 | 639268 | chr1_NT_456110v1_random 728 |
| 4828285 | 1683758 | 2214207 | | 33480 | 647481 | chr1_NT_456112v1_random 14017 |
| 1151154 | 1651020 | 2742270 | | 40363 | 500957 | chr1_NT_456115v1_random 24088 |
| 1273305 | 3827936 | 2582966 | | 3939 | 500957 | chr1_NT_456181v1_random 8431 |
| 638747 | 5273739 | 3610524 | | 65190 | 59043 | chr1_NT_456186v1_random 10030 |
| 4984994 | 5193105 | 1326585 | | 7843 | 979262 | chr1_NT_456276v1_random 12747 |
| 4660593 | 3763266 | 3560207 | | 76627 | 599753 | chr1_NT_456335v1_random 455 |
| 966956 | 3064262 | 3560207 | | 11975 | 1177471 | chr1_NT_456380v1_random 5977 |
| 1252863 | 1488659 | 1178166 | | 60883 | 502981 | chr1_NT_456410v1_random 2993 |
| 2363276 | 4195864 | 1073824 | | 40203 | 1596842 | chr1_NT_457707v1_random 641 |
| 5026718 | 4195864 | 4407751 | | 76608 | 75747 | chr1_NT_457707v1_random 851 |
| 1123568 | 5273835 | 2217143 | | 75569 | 548659 | chr1_NT_457825v1_random 369 |
| 1055337 | 3158586 | 2309883 | | 49581 | 712292 | chr2_NT_457893v1_random 3264 |
| 2483547 | 4921356 | 1475916 | | 75638 | 492018 | chr2_NT_457893v1_random 15417 |
| 2534437 | 593375 | 3886679 | | 20007 | 831395 | chr2_NT_457893v1_random 12257 |
| 4935899 | 4879806 | 1113632 | | 26226 | 1521125 | chr2_NT_457893v1_random 3035 |
| 4797752 | 4731603 | 3152342 | | 77631 | 786884 | chr2_NT_457893v1_random 14858 |
| 1505957 | 5059615 | 1113643 | | 12044 | 679069 | chr2_NT_457893v1_random 6515 |
| 5186537 | 4731050 | 2714857 | | 71054 | 695293 | chr2_NT_457893v1_random 15291 |
| 1171393 | 4813757 | 1326635 | | 21394 | 1606604 | chr2_NT_457893v1_random 10541 |
| 613274 | 4195869 | 1967756 | | 74249 | 872657 | chr2_NT_457893v1_random 9214 |
| 1248487 | 1422005 | 3396207 | | 77596 | 1031154 | chr2_NT_457893v1_random 21375 |
| 1191526 | 1703732 | 3015704 | | 76292 | 1599478 | chr2_NT_458177v1_random 8997 |
| 459139 | 777099 | 1326631 | | 47354 | 341309 | chr2_NT_458177v1_random 6280 |
| 1137543 | 5229133 | 2163542 | | | 798434 | chr2_NT_458177v1_random 8995 |
| 979359 | 523786 | 3291454 | | | 1075676 | chr2_NT_458177v1_random 8995 |
| 1138411 | 4229521 | 3640080 | | | 514805 | chr2_NT_458493v1_random 5539 |
| 1323995 | 5276731 | 4056324 | | | 1607024 | chr3_NT_459782v1_random 1423 |
| 1505994 | 3463363 | 2946384 | | | 646975 | chr3_NT_459782v1_random 957 |
| 3454288 | 1483377 | 2486660 | | | 1606671 | chr3_NT_459789v1_random 266 |
| 1206302 | 4192357 | 2486660 | | | 1592818 | chr3_NT_459789v1_random 256 |
| 926798 | 4658588 | 3066444 | | | 1120223 | chr3_NT_459796v1_random 596 |
| 4806406 | 3336789 | 2486662 | | | 239044 | chr4_NT_459800v1_random 331914 |
| 1276829 | 2873044 | 4352015 | | | 783143 | chr4_NT_459800v1_random 233778 |
| 649320 | 4358945 | 2659664 | | | 872654 | chr4_NT_459800v1_random 331924 |

TABLE 3a
Representative chicken gene CpG sites on the multispecies DNA-methylation bead array
chip. Table shows an example 432 of the total 11956 chicken gene CpG sites.

| Chrom. 1 | Chrom. 2 | Chrom. 3 | Chrom. 4 | Chrom. 5 | Chrom. 6 | Chrom. 7 | Chrom. 8 | Chrom. 9 | Chrom. 10 | Chrom. 11 | Chrom. 12 |
|---|---|---|---|---|---|---|---|---|---|---|---|
| 67101188 | 33339556 | 67104445 | 25199816 | 8307060 | 8403537 | 15614107 | 16808418 | 8397811 | 8303614 | 1046708 | 8380218 |
| 67101199 | 33339595 | 67104470 | 25199953 | 8307062 | 8403555 | 15614127 | 16809270 | 8397825 | 8303735 | 1047036 | 8380398 |
| 185628233 | 6318873 | 25201811 | 75766196 | 16776415 | 16768792 | 21994780 | 1140026 | 5257096 | 16838099 | 6293270 | 5244166 |
| 185628419 | 6318892 | 25201834 | 75766199 | 16776474 | 16769037 | 21994838 | 1140036 | 5257099 | 16838223 | 6293272 | 5244177 |
| 42226884 | 9461167 | 2106043 | 1055486 | 25165899 | 25417011 | 26218138 | 2094923 | 6086347 | 3136096 | 10578272 | 14659307 |
| 42226907 | 9461207 | 2106059 | 1055508 | 25165914 | 25417033 | 26218141 | 2094933 | 6086588 | 3136113 | 10578303 | 14659621 |
| 186756879 | 23984846 | 9522342 | 12598143 | 50325256 | 5237568 | 29524534 | 4176787 | 11558558 | 12593019 | 14260125 | 634761 |
| 186757033 | 23984864 | 9522363 | 12598146 | 50325284 | 5237578 | 29524545 | 4176789 | 11558574 | 12593068 | 14260139 | 635015 |
| 43976934 | 26092671 | 10483708 | 17839519 | 2085803 | 11529765 | 520600 | 5325476 | 19084501 | 14628768 | 17827326 | 1580614 |
| 43996631 | 26092774 | 10483915 | 17839645 | 2085956 | 11529884 | 520604 | 5326254 | 19084560 | 14628881 | 17827446 | 1580728 |
| 43996647 | 32560017 | 14661005 | 31480271 | 7331308 | 17824703 | 2760948 | 12568258 | 19920585 | 17827817 | 18881661 | 2574812 |
| 44596794 | 32560055 | 14661121 | 31480371 | 12587441 | 17824809 | 2761006 | 12568281 | 19920713 | 17827866 | 18881701 | 2574827 |
| 44597115 | 34591814 | 20967709 | 37627872 | 12587511 | 20984464 | 3693191 | 22008501 | 1308316 | 19884928 | 390144 | 2782954 |
| 44664923 | 34591816 | 20968094 | 37627884 | 13646506 | 20984488 | 3693302 | 22008517 | 1308479 | 19884948 | 390167 | 2782967 |
| 44665586 | 36792299 | 23024829 | 38713711 | 13646512 | 24085106 | 4813249 | 24134871 | 1715695 | 2764771 | 771739 | 2869740 |
| 44860363 | 36792302 | 23025056 | 38714002 | 14662137 | 24085112 | 4813373 | 24134874 | 1715697 | 2764796 | 771757 | 2869744 |
| 44860613 | 37786439 | 26263105 | 46163248 | 14662187 | 26205929 | 5781975 | 26016443 | 2847556 | 2929886 | 1456405 | 4381456 |
| 188765887 | 37786525 | 26263208 | 46163338 | 18900698 | 26205947 | 5782475 | 26016921 | 2848060 | 2929930 | 1456408 | 4603024 |
| 188765890 | 43012011 | 31547141 | 54482428 | 18900846 | 786736 | 7139833 | 27133173 | 3661463 | 3667377 | 1604013 | 4603037 |
| 45152826 | 43012143 | 31547210 | 54482525 | 35104985 | 4326952 | 7139843 | 27133426 | 3661539 | 3667577 | 1604050 | 4966098 |
| 45152832 | 47181145 | 32528162 | 60880221 | 35104996 | 4327003 | 8335782 | 28325453 | 4589620 | 4286271 | 2009111 | 4966524 |
| 45315904 | 47181153 | 32528255 | 60880349 | 37767357 | 4455306 | 8335807 | 28325567 | 4589624 | 4286726 | 2009116 | 5682607 |
| 45315910 | 55593834 | 42987388 | 68113638 | 37767364 | 4455327 | 9938239 | 49798 | 4714832 | 4625334 | 2644622 | 5682693 |
| 45534485 | 55593876 | 42987393 | 68113644 | 38680621 | 8745687 | 9938291 | 49802 | 4715030 | 4625353 | 2644691 | 6578283 |
| 45534521 | 61780834 | 44610084 | 69138370 | 38680909 | 8745725 | 10954989 | 827384 | 5525906 | 5753844 | 3733699 | 6578285 |
| 45668702 | 61780837 | 44610103 | 69138470 | 45115466 | 9169856 | 10955047 | 827438 | 5526011 | 5754391 | 3733956 | 8759965 |
| 45668759 | 68102167 | 46068059 | 70266891 | 45115470 | 9170093 | 11182930 | 1445934 | 5648936 | 5937118 | 5112002 | 8760061 |
| 45718413 | 68102190 | 46068120 | 70266893 | 48237445 | 9840784 | 11183028 | 1445937 | 5649217 | 5937149 | 5112222 | 8863206 |
| 45718438 | 74530514 | 48217116 | 74249635 | 48237567 | 9840808 | 11248389 | 2703905 | 5898860 | 6151181 | 5366687 | 8863219 |
| 45775466 | 74530670 | 48217173 | 74249658 | 141245 | 10627134 | 11248416 | 2703967 | 5898862 | 6151202 | 5366729 | 9778045 |
| 45775639 | 78648499 | 49135422 | 80579166 | 141294 | 10627401 | 11631892 | 3498965 | 7723994 | 6646118 | 6468133 | 9778149 |
| 195117496 | 78648510 | 49135605 | 80579169 | 658536 | 10876195 | 11631974 | 3498975 | 7724095 | 6646708 | 6468152 | 10720113 |
| 195117499 | 84949816 | 55607400 | 81756143 | 658567 | 10876209 | 11635481 | 3802109 | 8025583 | 6959256 | 6819127 | 10720125 |
| 46668862 | 84949820 | 55607488 | 81756191 | 1446316 | 11048159 | 11635584 | 3802116 | 8025588 | 6959340 | 6819133 | 10991823 |
| 46668867 | 98536503 | 66132431 | 82821003 | 1446320 | 11048191 | 12318230 | 4710320 | 8127126 | 7045099 | 7495593 | 10991883 |
| 46695191 | 98536521 | 66132498 | 82821016 | 2932698 | 11272720 | 12318270 | 4710336 | 8127231 | 7045102 | 7495992 | 11256589 |

TABLE 3b
Representative chicken gene CpG sites on the multispecies DNA-methylation bead array
chip. Table shows an example 426 of the total 11956 chicken gene CpG sites.

| Chrom. 13 | Chrom. 14 | Chrom. 15 | Chrom. 16 | Chrom. 17 | Chrom. 18 | Chrom. 19 | Chrom. 20 | Chrom. 21 | Chrom. 22 | Chrom. 23 | Chrom. 24 |
|---|---|---|---|---|---|---|---|---|---|---|---|
| 8416006 | 2120134 | 3160307 | 17094 | 8376726 | 3145938 | 3142824 | 8397786 | 3148062 | 1423101 | 1704685 | 976876 |
| 8416270 | 2120174 | 3160349 | 17122 | 8376779 | 3145944 | 3142909 | 8397789 | 3148067 | 1423108 | 1704715 | 976902 |
| 16771880 | 7328145 | 5247982 | 72917 | 1055012 | 5377038 | 657590 | 2968929 | 142138 | 2371295 | 1968677 | 1856333 |
| 16771889 | 7328157 | 5248005 | 72919 | 1055065 | 5377115 | 657747 | 2968972 | 142144 | 2371309 | 1968753 | 1856359 |
| 1049758 | 11663496 | 6272683 | 80815 | 2079871 | 9554000 | 1258827 | 13629186 | 520362 | 2749775 | 2363682 | 3192495 |
| 1049811 | 11663498 | 6272713 | 80818 | 2079891 | 9554072 | 1258841 | 13629204 | 520389 | 2750000 | 2363711 | 3192578 |
| 10536415 | 12622765 | 9438545 | 93134 | 3187582 | 540440 | 1957159 | 409663 | 663684 | 3524175 | 2595800 | 4193030 |
| 10536462 | 12622769 | 9438560 | 93344 | 3187684 | 540541 | 1957188 | 409703 | 663690 | 4053794 | 2595803 | 4193147 |
| 912015 | 14701463 | 241922 | 104860 | 5197989 | 734942 | 2858627 | 949165 | 905095 | 4053796 | 3534940 | 125375 |
| 912484 | 14702395 | 242095 | 104873 | 5198007 | 734956 | 2858846 | 949281 | 905099 | 4729245 | 3534971 | 125406 |
| 1194829 | 303170 | 1315539 | 115888 | 10452144 | 930312 | 3409420 | 1161275 | 2373480 | 4729247 | 4056057 | 394987 |
| 1194832 | 303172 | 1315969 | 115932 | 10452161 | 930316 | 3409648 | 1161472 | 2373485 | 101155 | 4056063 | 394995 |
| 1543237 | 1318196 | 2765588 | 150791 | 941659 | 1855723 | 3484115 | 1773373 | 2765990 | 101701 | 4325868 | 1572445 |
| 1543256 | 1318201 | 2765598 | 151339 | 941661 | 1855777 | 3484134 | 1773845 | 2766029 | 246447 | 4325964 | 1572532 |
| 2836441 | 2624552 | 3336237 | 164366 | 1438746 | 2352191 | 3851682 | 2241974 | 2876779 | 246516 | 5355467 | 2367106 |
| 2836577 | 2624654 | 3336265 | 164379 | 1438751 | 2352470 | 3851759 | 2242415 | 2876785 | 283842 | 5355512 | 2367110 |
| 4122460 | 2743954 | 3525790 | 168804 | 1678870 | 3380520 | 4465489 | 3800612 | 3393774 | 283901 | 5768059 | 2882333 |
| 4122468 | 2744164 | 3526061 | 168862 | 1679002 | 3380619 | 4465606 | 3801074 | 3393787 | 312743 | 5768189 | 2882462 |
| 4707018 | 3315861 | 3784415 | 177532 | 1971456 | 4577940 | 5070534 | 3978395 | 3549789 | 312761 | 28267 | 3557024 |
| 4707161 | 3315866 | 3784453 | 177604 | 1971461 | 4577965 | 5070543 | 3978445 | 3550002 | 348152 | 28296 | 3557092 |
| 7127937 | 3417578 | 4430869 | 178871 | 2213124 | 4832339 | 5494940 | 5500235 | 3735042 | 348184 | 205656 | 5111792 |
| 7127947 | 3417580 | 4431030 | 178885 | 2213278 | 4832453 | 5494942 | 5500252 | 3735044 | 424097 | 205680 | 5111794 |
| 7849364 | 3530153 | 4634790 | 190978 | 2600916 | 4972395 | 5635063 | 6166919 | 4066712 | 424131 | 229897 | 5779860 |
| 7849425 | 3530155 | 4634797 | 191103 | 2600938 | 4972738 | 5635083 | 6167188 | 4066720 | 441988 | 229920 | 5779878 |
| 7895863 | 4690169 | 4891573 | 210756 | 3718965 | 5103908 | 5768580 | 7426774 | 5072981 | 442172 | 240549 | 22822 |
| 7896124 | 4690171 | 4891613 | 210963 | 3719380 | 5103916 | 5768840 | 7426817 | 5073002 | 542903 | 240568 | 22837 |
| 8008633 | 4854394 | 4984456 | 224382 | 5368127 | 5885896 | 6160911 | 7900225 | 5512552 | 543015 | 284729 | 34809 |
| 8008655 | 5120990 | 4984465 | 224385 | 5368219 | 5885920 | 6160952 | 7900313 | 5512792 | 824340 | 284943 | 34837 |
| 8651174 | 5121076 | 5385230 | 294467 | 5618391 | 6381996 | 6574248 | 7993223 | 6063423 | 824389 | 579261 | 47044 |
| 8651182 | 6811908 | 5385238 | 294486 | 5618397 | 6382019 | 6574382 | 7993347 | 6063513 | 825221 | 579271 | 47048 |
| 8748023 | 6811923 | 5907804 | | 7111518 | 6940249 | 6937888 | 9025018 | 6428053 | 825224 | 578579 | 57731 |
| 8748040 | 7462952 | 5907987 | | 7111588 | 6940253 | 6937917 | 9025021 | 6428151 | 951398 | 578593 | 57787 |
| 9162161 | 7463006 | 6035194 | | 7622794 | 7214580 | 7971723 | 9688887 | 6549349 | 951638 | 596615 | 158732 |
| 9162165 | 7599414 | 6035220 | | 7622796 | 7214706 | 7971731 | 9688892 | 6549360 | 1219272 | 596621 | 158869 |
| 10621961 | 7599570 | 6162585 | | 7909303 | 9058496 | 8136377 | 9829695 | 143185 | 1219283 | 1264849 | 221561 |
| 10622135 | 8522661 | 6162591 | | 7909422 | 9058506 | 8136387 | 9829707 | 191817 | 1258840 | 1264860 | 221572 |

TABLE 3c
Representative chicken gene CpG sites on the multispecies DNA-methylation bead array
chip. Table shows an example 229 of the total 11956 chicken gene CpG sites.

| Chrom. 25 | Chrom. 26 | Chrom. 27 | Chrom. 28 | Chrom. 30 | Chrom. 32 | Chrom. 33 | Scaffolds |
|---|---|---|---|---|---|---|---|
| 123610 | 1054737 | 3146878 | 135754 | 10419 | 12931 | 251123 | chr4_NT_459801v1_random 24123 |
| 123687 | 1054819 | 3146913 | 136468 | 10424 | 12934 | 251452 | chr4_NT_459801v1_random 24148 |
| 901305 | 2094274 | 5249967 | 261381 | 24862 | 50116 | 361587 | chr4_NT_459801v1_random 25200 |
| 901377 | 2094300 | 5250009 | 261395 | | 50329 | 361597 | chr4_NT_459801v1_random 25270 |
| 1442331 | 5221177 | 1573963 | 1294660 | | 60470 | 645915 | chr4_NT_459801v1_random 91794 |
| 1442334 | 5221401 | 1573966 | 1294826 | | 60495 | 645952 | chr4_NT_459801v1_random 91803 |
| 1582756 | 137321 | 2681014 | 1447791 | | 75352 | 6730 | chr4_NT_459801v1_random 90879 |
| 1582769 | 137369 | 2681150 | 1448057 | | 75441 | 7075 | chr4_NT_459801v1_random 90893 |
| 1700604 | 369783 | 3672662 | 2496145 | | 74763 | 17039 | chr4_NT_459801v1_random 110230 |
| 1700606 | 369828 | 3672665 | 2496320 | | 74772 | 17042 | chr4_NT_459801v1_random 110232 |
| 2482631 | 617091 | 4455719 | 2874174 | | | 32227 | chr4_NT_459801v1_random 131175 |
| 2482633 | 617104 | 4455730 | 2874261 | | | 32230 | chr4_NT_459801v1_random 131182 |
| 24593 | 925182 | 4719283 | 3271407 | | | 137304 | chr4_NT_459801v1_random 160959 |
| 24628 | 925201 | 4719323 | 3271450 | | | 137307 | chr4_NT_459801v1_random 160964 |
| 32279 | 1178811 | 4856706 | 3532462 | | | 290519 | chr4_NT_459802v1_random 17025 |
| 32281 | 1178818 | 4856710 | 3532474 | | | 290576 | chr4_NT_459802v1_random 17028 |
| 44413 | 2233140 | 5374149 | 3799795 | | | 293669 | chr4_NT_459802v1_random 27083 |
| 44449 | 2233167 | 5374261 | 3800242 | | | 293685 | chr4_NT_459802v1_random 27094 |
| 82854 | 2617264 | 171323 | 3909633 | | | 308010 | chr5_NT_460419v1_random 11618 |
| 82857 | 2617280 | 171361 | 3909664 | | | 308012 | chr5_NT_460419v1_random 11740 |
| 110767 | 2844689 | 1211446 | 4065543 | | | 454936 | chr5_NT_460419v1_random 24818 |
| 110989 | 2844722 | 1211631 | 4065554 | | | 454948 | chr5_NT_460419v1_random 24825 |
| 814289 | 3935300 | 1370375 | 4858019 | | | 464695 | chr5_NT_460421v1_random 8022 |
| 814296 | 3935304 | 1370445 | 4858380 | | | 464702 | chr5_NT_460421v1_random 8027 |
| 932691 | 4440144 | 1399278 | 29443 | | | 625698 | chr5_NT_460421v1_random 29586 |
| 932703 | 4440153 | 1399295 | 495550 | | | 625731 | chr5_NT_460421v1_random 29732 |
| 976180 | 4583760 | 1478413 | 495765 | | | 675493 | chr7_NT_461079v1_random 12093 |
| 976273 | 4583764 | 1478660 | 521992 | | | 675540 | chr9_NT_461630v1_random 7531 |
| 1200242 | 4710196 | 1496691 | 521995 | | | 678142 | chr19_NT_462742v1_random 5663 |
| 1200290 | 4710233 | 1496716 | 616313 | | | 678201 | chr19_NT_462742v1_random 5665 |
| 1284725 | 34961 | 1495772 | 616318 | | | 712292 | chr22_NT_463002v1_random 4198 |
| 1284735 | 35043 | 1495804 | 649204 | | | 712419 | chr22_NT_463002v1_random 4209 |
| 1335347 | 47967 | 1516370 | 649301 | | | 715911 | chr22_NT_463002v1_random 13669 |
| 1335355 | 47970 | 1516393 | 661304 | | | 715916 | chr22_NT_463002v1_random 13734 |
| 1356843 | 93874 | 1523513 | 661307 | | | 753646 | chr22_NT_463003v1_random 23747 |
| 1356886 | 93895 | 1523554 | 700723 | | | 753656 | chr22_NT_463003v1_random 23782 |

TABLE 4a
Representative CpG sites from the lowly methylated regions of the chicken
genome on the multispecies DNA-methylation bead array chip. Table shows an
example 309 of the total 3883 chicken lowly methylated region CpG sites.

| Chrom. 1 | Chrom. 2 | Chrom. 3 | Chrom. 4 | Chrom. 5 | Chrom. 6 | Chrom. 7 |
|---|---|---|---|---|---|---|
| 1144371 | 25436543 | 27449584 | 1489571 | 840052 | 8416247 | 2418250 |
| 1144422 | 25436609 | 27449598 | 1489598 | 1942053 | 8416279 | 2418424 |
| 1144434 | 25437175 | 27449601 | 1489792 | 1942092 | 8416282 | 2418573 |
| 1144440 | 25437356 | 67444312 | 1489971 | 1942115 | 8416337 | |
| 1145145 | 25437685 | 67444323 | 1489974 | 1942133 | 8416393 | |
| 1145368 | 31315303 | 67444382 | 1490028 | 1942148 | 8416426 | |
| 1145401 | 31315336 | 67444417 | 1490059 | 1942157 | 8416432 | |
| 1145535 | 31315509 | 67444579 | 1490083 | 1942165 | 8416437 | |
| 1145599 | 31315527 | 101470013 | 1490094 | 1942276 | 8416503 | |
| 1145624 | 31315610 | 101470036 | 1490113 | 1942352 | 8416518 | |
| 1145689 | 31315727 | 101470041 | 1490184 | 1942404 | 8416528 | |
| 1145818 | 31315732 | 101470050 | 1490232 | 1942440 | 8416584 | |
| 1145895 | 31315777 | | 1490286 | 1942468 | 8416588 | |
| 1145902 | 31316281 | | 1490355 | 1942488 | | |
| 1537687 | 31316293 | | 1490465 | 1942725 | | |
| 1537693 | 31316354 | | 1490569 | 1942749 | | |
| 1538038 | 31316368 | | 1490684 | 1942804 | | |
| 1679706 | 31991773 | | 2049225 | 1942823 | | |
| 1679815 | 91174564 | | 2049417 | 1942842 | | |
| 3311010 | 91174576 | | 2049488 | 33274964 | | |
| 3311013 | 91174947 | | 2049615 | 33275170 | | |
| 3311017 | 91175021 | | 2434501 | 33275629 | | |
| 3311023 | 91175027 | | 2434525 | 33275638 | | |
| 3400955 | 121135304 | | 2434576 | 33275641 | | |
| 3400985 | 146200847 | | 2434582 | 33275649 | | |
| 3400987 | 146200905 | | 2434629 | 33275717 | | |
| 3401010 | 146200966 | | 2434642 | 33275745 | | |
| 3401012 | | | 2434679 | 33275754 | | |
| 3401032 | | | 7321073 | 33275777 | | |
| 3402804 | | | 7321318 | 33275794 | | |
| 3402856 | | | 8453317 | 33275864 | | |
| 3406325 | | | 8453340 | 33276231 | | |
| 3406566 | | | 8453366 | 33276328 | | |
| 3406668 | | | 8453396 | 33276348 | | |
| 3420046 | | | 8453599 | 33276350 | | |

| Chrom. 8 | Chrom. 9 | Chrom. 10 | Chrom. 11 | Chrom. 12 | Chrom. 13 |
|---|---|---|---|---|---|
| 13056205 | 23812504 | 1521168 | 675301 | 1423910 | 988844 |
| | 23812559 | 4090293 | 675389 | 1424097 | 988848 |
| | 23812678 | 4090310 | 675397 | 1880827 | 988920 |
| | | 4090317 | 675399 | 1880859 | 988939 |
| | | 4090346 | 675405 | 2424809 | 989024 |
| | | 4118063 | 675474 | 2424826 | 989178 |
| | | 4118148 | 675512 | 2424891 | 1505896 |
| | | 4118233 | 675517 | 2424962 | 1506727 |
| | | 4118331 | 675525 | 2424964 | 2263241 |
| | | 4118377 | 1320040 | 2424987 | 2263255 |
| | | 4118414 | 1320102 | 2425024 | 2263500 |
| | | 4118590 | 1320137 | 2425130 | 2263505 |
| | | 4118596 | 1320164 | 2425133 | 2263509 |
| | | 4118682 | 1320185 | 3227813 | 2263526 |
| | | 4118708 | 1320246 | 3227885 | 2263556 |
| | | 4118757 | 1899284 | 3251705 | 5919931 |
| | | 4118766 | 4104113 | 3251741 | 5920662 |
| | | 4118777 | 4104281 | 3251755 | 5920908 |
| | | 4118856 | 4104299 | 3251778 | 8094372 |
| | | 4118922 | 4104360 | 3251800 | 8094576 |
| | | 4118971 | 4104386 | 3251861 | 8094688 |
| | | 4119095 | 4643108 | 3251867 | 8094816 |
| | | 4316177 | 4643121 | 3251956 | 8363158 |
| | | 4316180 | 4643127 | 3251978 | 8363197 |
| | | 4316254 | 4643133 | 3252015 | 8363260 |
| | | 4936479 | 4643147 | 3252062 | 8363270 |
| | | 4936514 | 4643150 | 3252134 | 8363519 |
| | | 4936546 | 4643158 | 3252153 | 8363542 |
| | | 4936743 | 7052776 | 3358963 | 8363741 |
| | | 5227051 | 7052862 | 3359221 | 8363754 |
| | | 5227375 | 7053020 | 3359439 | 8363819 |
| | | 5227381 | 7053082 | 3359558 | 8363900 |
| | | 5227390 | 7053112 | 3359601 | 8364015 |
| | | 5478439 | 8513603 | 3359682 | 8364065 |
| | | 5478496 | 8513612 | 4485087 | 8364116 |

TABLE 4b
Representative CpG sites from the lowly methylated regions of the chicken genome on the multispecies DNA-methylation
bead array chip. Table shows an example 391 of the total 3883 chicken lowly methylated region CpG sites.

| Chrom. 14 | Chrom. 15 | Chrom. 17 | Chrom. 18 | Chrom. 19 | Chrom. 20 | Chrom. 21 | Chrom. 22 | Chrom. 23 | Chrom. 25 | Chrom. 26 | Chrom. 27 |
|---|---|---|---|---|---|---|---|---|---|---|---|
| 717753 | 326616 | 1487989 | 2050242 | 373694 | 252409 | 179365 | 51084 | 28707 | 1101311 | 438619 | 1563746 |
| 718044 | 327083 | 1488007 | 2050321 | 373709 | 252422 | 179502 | 51259 | 28788 | 1101351 | 438673 | 1563751 |
| 718104 | 629756 | 1488019 | 2050354 | 373760 | 252432 | 179566 | 51303 | 28837 | 1101361 | 438693 | 1563792 |
| 718183 | 629901 | 1488030 | 2050360 | 373790 | 252438 | 869541 | 51322 | 28944 | 2216100 | 438698 | 1563873 |
| 718223 | 713219 | 1488095 | 2050467 | 373805 | 318975 | 869554 | 51338 | 28971 | 2444748 | 438753 | 1563927 |
| 718363 | 713254 | 1488308 | 2050472 | 373831 | 1025009 | 869632 | 51539 | 114992 | 2444805 | 438773 | 1563931 |
| 718373 | 713289 | 1488387 | 2050582 | 373896 | 1025099 | 869660 | 51630 | 115061 | | 438792 | 1563940 |
| 718385 | 713335 | 1488502 | 2050616 | 374005 | 1025106 | 985281 | 528526 | 115114 | | 438867 | 1563976 |
| 718535 | 713413 | 1488510 | 2050724 | 374035 | 1025139 | 985303 | 528585 | 115135 | | 439295 | 1564049 |
| 866887 | 713425 | 1488774 | 2094353 | 374061 | 1025300 | 985355 | 528595 | 115156 | | 439374 | 1564119 |
| 866911 | 713437 | 1488788 | 2094425 | 374112 | 1025310 | 1035884 | 528607 | 115163 | | 439384 | 1597344 |
| 866973 | 713439 | 1758192 | 2094436 | 374209 | 1025498 | 1035909 | 528655 | 115234 | | 439408 | 1597405 |
| 867007 | 713447 | 1758219 | 2094476 | 374286 | 3497125 | 1035937 | 528695 | 230512 | | 439510 | 1597424 |
| 867146 | 713523 | 1802783 | 2094546 | 374360 | 3497148 | 1035946 | 528750 | 230609 | | 633381 | 1597442 |
| 867219 | 713530 | 1802821 | 2094556 | 374379 | 3497168 | 1035991 | 528777 | 230618 | | 633400 | 1597446 |
| 867322 | 713544 | 1802861 | 2094572 | 374417 | 3497178 | 1036074 | 528804 | 230676 | | 633404 | 1597452 |
| 867353 | 764767 | 1802910 | 2260453 | 655961 | 3497187 | 1036143 | 528902 | 230707 | | 633475 | 1597456 |
| 867443 | 764811 | 1802921 | 2260462 | 655963 | 3497231 | 1036168 | 528923 | 806809 | | 633502 | 1597577 |
| 867552 | 764816 | 2457684 | 2260502 | 656002 | 3497237 | 1685237 | 529005 | 806834 | | 680295 | 1597722 |
| 867622 | 764870 | 2457693 | 2358528 | 656037 | 3497240 | 1685288 | 529098 | 953515 | | 680341 | 1597730 |
| 1156459 | 764874 | 2457705 | 2358653 | 656070 | 3497256 | 1685366 | 529137 | 953588 | | 680348 | 1597734 |
| 1156538 | 764879 | 2457707 | 2358665 | 656075 | 3497277 | 1685443 | 529358 | 953606 | | 680374 | 1597754 |
| 1156554 | 764894 | 2457714 | 2358772 | 1258121 | 3497279 | 1685492 | 1063445 | 953775 | | 1116234 | 1597774 |
| 1156600 | 764943 | 2457781 | 2358859 | 1258154 | 5250773 | 1685494 | 1063506 | 953885 | | 1116257 | 1597806 |
| 1687955 | 764977 | 2457829 | 2358910 | 1384385 | 5250776 | 1685497 | 1063656 | 953992 | | 1116266 | 1597809 |
| 1688170 | 765010 | 2457842 | 2359041 | 1384453 | 5250818 | 1685609 | 1063746 | 953997 | | 1116281 | 3073625 |
| 1711913 | 1471202 | 2457850 | 2359144 | 1384478 | 5250884 | 1685611 | 1414543 | 954041 | | 1116361 | 3073646 |
| 1711921 | 1471306 | 2457891 | 2359159 | 1384489 | 5251046 | 1685765 | 1414571 | 954066 | | 1116402 | 3073681 |
| 1711954 | 1471316 | 2457911 | 2359239 | 1384521 | 5536944 | 1685788 | 1414623 | 954119 | | 1116420 | 3073693 |
| 1711985 | 1471327 | 2458002 | 2359272 | 1384529 | 5536965 | 1685846 | 1414639 | 954134 | | 1116437 | 3073703 |
| 1712020 | 1471344 | 2458079 | 2359301 | 1384600 | 5537292 | 1685863 | 1414654 | 954145 | | 1116455 | 3073823 |
| 1712033 | 1538374 | 2458103 | 2359551 | 1384621 | 5537307 | 1696755 | 1414682 | 954238 | | 1116469 | 3073909 |
| 1712112 | 1538481 | 2458169 | 2359632 | 3101867 | 5537341 | 1696766 | 1414686 | 993136 | | 1116538 | 3074161 |
| 1712127 | 1538526 | 2458210 | 2855673 | 3102113 | 5537426 | 1696772 | 1414706 | 993198 | | 1116544 | 3074163 |
| 2151678 | 1538548 | 2458342 | 2855802 | 3102126 | 5537451 | 1696902 | 1414716 | 993226 | | 1116600 | 3074186 |

TABLE 5
Representative CpG sites from the CHO dynamic probes on the
multispecies DNA-methylation bead array chip. Table shows
an example 180 of the total 6827 CHO dynamic CpG sites.

| CpG Site Location | CpG Site Location | CpG Site Location |
|---|---|---|
| NW_003613580v1_1326952 | NW_003613677v1_485534 | NW_003613779v1_880351 |
| NW_003613581v1_6655590 | NW_003613678v1_281475 | NW_003613782v1_1746724 |
| NW_003613582v1_6232396 | NW_003613679v1_314864 | NW_003613783v1_458775 |
| NW_003613583v1_1599807 | NW_003613680v1_2280392 | NW_003613784v1_2017758 |
| NW_003613584v1_5610364 | NW_003613681v1_929551 | NW_003613785v1_1072505 |
| NW_003613585v1_5468989 | NW_003613683v1_842682 | NW_003613786v1_1481329 |
| NW_003613586v1_604683 | NW_003613684v1_841975 | NW_003613787v1_667388 |
| NW_003613587v1_1803863 | NW_003613685v1_1250629 | NW_003613788v1_1017962 |
| NW_003613588v1_5227342 | NW_003613686v1_502068 | NW_003613789v1_1384719 |
| NW_003613589v1_125351 | NW_003613687v1_2454906 | NW_003613790v1_1852643 |
| NW_003613590v1_390828 | NW_003613688v1_345243 | NW_003613792v1_1618002 |
| NW_003613591v1_3787346 | NW_003613689v1_895897 | NW_003613793v1_1360768 |
| NW_003613593v1_2622831 | NW_003613690v1_837465 | NW_003613794v1_1706765 |
| NW_003613594v1_1593451 | NW_003613691v1_547846 | NW_003613795v1_1554641 |
| NW_003613595v1_2437503 | NW_003613692v1_935490 | NW_003613796v1_1782577 |
| NW_003613596v1_1178980 | NW_003613694v1_1525189 | NW_003613797v1_368190 |
| NW_003613597v1_3738865 | NW_003613695v1_535441 | NW_003613798v1_1940800 |
| NW_003613598v1_428822 | NW_003613696v1_849439 | NW_003613799v1_880785 |
| NW_003613599v1_4162490 | NW_003613697v1_2627054 | NW_003613800v1_2027635 |
| NW_003613600v1_4296777 | NW_003613698v1_35286 | NW_003613801v1_1986099 |
| NW_003613601v1_4232113 | NW_003613699v1_2094756 | NW_003613802v1_1946689 |
| NW_003613602v1_2660615 | NW_003613700v1_1645691 | NW_003613803v1_2005692 |
| NW_003613603v1_3009027 | NW_003613701v1_467342 | NW_003613804v1_1992909 |
| NW_003613604v1_4568838 | NW_003613702v1_2406468 | NW_003613805v1_54058 |
| NW_003613605v1_4103939 | NW_003613703v1_1665676 | NW_003613806v1_796951 |
| NW_003613606v1_3526150 | NW_003613704v1_2283408 | NW_003613807v1_1454340 |
| NW_003613607v1_494352 | NW_003613705v1_477606 | NW_003613808v1_70243 |
| NW_003613608v1_1461656 | NW_003613706v1_403846 | NW_003613809v1_19092 |
| NW_003613609v1_618885 | NW_003613707v1_1417912 | NW_003613810v1_726530 |
| NW_003613610v1_48254 | NW_003613708v1_2285548 | NW_003613811v1_1120498 |
| NW_003613611v1_3905959 | NW_003613709v1_1859433 | NW_003613815v1_1088955 |
| NW_003613612v1_2474397 | NW_003613710v1_939115 | NW_003613816v1_1861042 |
| NW_003613613v1_792911 | NW_003613711v1_1133968 | NW_003613817v1_971871 |
| NW_003613614v1_442749 | NW_003613713v1_2484897 | NW_003613818v1_1200241 |
| NW_003613615v1_611781 | NW_003613714v1_1181079 | NW_003613820v1_959259 |
| NW_003613616v1_3607474 | NW_003613715v1_1975884 | NW_003613821v1_1586301 |
| | CpG Site Location | CpG Site Location |
| | NW_003613886v1_925106 | NW_003613999v1_703212 |
| | NW_003613887v1_1147996 | NW_003614002v1_962191 |
| | NW_003613888v1_1017586 | NW_003614003v1_169457 |
| | NW_003613889v1_1417212 | NW_003614004v1_1288742 |
| | NW_003613890v1_801604 | NW_003614007v1_503038 |
| | NW_003613891v1_1262447 | NW_003614009v1_121347 |
| | NW_003613893v1_1498090 | NW_003614011v1_928277 |
| | NW_003613894v1_412692 | NW_003614012v1_647499 |
| | NW_003613896v1_115980 | NW_003614013v1_900678 |
| | NW_003613898v1_1404559 | NW_003614014v1_133024 |
| | NW_003613899v1_957263 | NW_003614015v1_615547 |
| | NW_003613900v1_824846 | NW_003614016v1_846601 |
| | NW_003613901v1_1224107 | NW_003614017v1_749160 |
| | NW_003613904v1_1371616 | NW_003614018v1_227156 |
| | NW_003613905v1_451154 | NW_003614019v1_115334 |
| | NW_003613906v1_592199 | NW_003614021v1_47735 |
| | NW_003613907v1_677528 | NW_003614022v1_619404 |
| | NW_003613908v1_498033 | NW_003614023v1_178029 |
| | NW_003613909v1_1646021 | NW_003614024v1_1124338 |
| | NW_003613910v1_156940 | NW_003614025v1_1347843 |
| | NW_003613911v1_949426 | NW_003614028v1_1106361 |
| | NW_003613912v1_304890 | NW_003614031v1_277986 |
| | NW_003613913v1_1071537 | NW_003614033v1_175922 |
| | NW_003613918v1_1417318 | NW_003614034v1_919614 |
| | NW_003613919v1_1169864 | NW_003614036v1_467131 |
| | NW_003613920v1_770607 | NW_003614037v1_215971 |
| | NW_003613921v1_1256651 | NW_003614039v1_1166581 |
| | NW_003613922v1_998249 | NW_003614040v1_517892 |
| | NW_003613923v1_933797 | NW_003614042v1_516454 |
| | NW_003613925v1_117878 | NW_003614045v1_99013 |
| | NW_003613926v1_1436324 | NW_003614046v1_266088 |
| | NW_003613927v1_1243314 | NW_003614047v1_186714 |
| | NW_003613928v1_256531 | NW_003614048v1_583899 |
| | NW_003613930v1_410410 | NW_003614050v1_503952 |
| | NW_003613933v1_1506415 | NW_003614051v1_1226799 |
| | NW_003613934v1_860354 | NW_003614054v1_1020048 |

TABLE 6
Representative CHO gene specific CpG sites on the multispecies
DNA-methylation bead array chip. Table shows an example
180 of the total 15300 CHO gene specific CpG sites.

| CpG Site Location | CpG Site Location | CpG Site Location |
|---|---|---|
| chrM_813 | NW_003613675v1_260874 | NW_003613771v1_931811 |
| NW_003613580v1_6362145 | NW_003613676v1_932803 | NW_003613772v1_119151 |
| NW_003613581v1_2129554 | NW_003613677v1_2089973 | NW_003613773v1_769467 |
| NW_003613582v1_6304430 | NW_003613678v1_8626 | NW_003613774v1_593289 |
| NW_003613583v1_6052067 | NW_003613679v1_1775675 | NW_003613775v1_1504427 |
| NW_003613584v1_1804907 | NW_003613681v1_2196196 | NW_003613777v1_1907578 |
| NW_003613585v1_1483139 | NW_003613682v1_1931609 | NW_003613778v1_186251 |
| NW_003613586v1_2487681 | NW_003613683v1_2738024 | NW_003613779v1_121228 |
| NW_003613587v1_3711394 | NW_003613684v1_1835908 | NW_003613780v1_383585 |
| NW_003613588v1_4150348 | NW_003613685v1_2071671 | NW_003613781v1_1386678 |
| NW_003613589v1_2109309 | NW_003613686v1_2197277 | NW_003613782v1_658312 |
| NW_003613591v1_4480749 | NW_003613687v1_2599221 | NW_003613783v1_876243 |
| NW_003613592v1_2130751 | NW_003613688v1_2330993 | NW_003613784v1_994724 |
| NW_003613593v1_3635541 | NW_003613689v1_2629015 | NW_003613785v1_332786 |
| NW_003613594v1_2164463 | NW_003613690v1_2288872 | NW_003613786v1_742832 |
| NW_003613595v1_4564812 | NW_003613691v1_1687095 | NW_003613787v1_1696166 |
| NW_003613596v1_1892565 | NW_003613692v1_276111 | NW_003613788v1_1088057 |
| NW_003613597v1_4137215 | NW_003613693v1_1862765 | NW_003613789v1_2042610 |
| NW_003613598v1_471636 | NW_003613694v1_1103877 | NW_003613790v1_1298678 |
| NW_003613599v1_4572940 | NW_003613695v1_2305382 | NW_003613791v1_1836693 |
| NW_003613600v1_3978084 | NW_003613696v1_1171613 | NW_003613792v1_786014 |
| NW_003613601v1_4548253 | NW_003613697v1_2266433 | NW_003613793v1_206634 |
| NW_003613603v1_2593449 | NW_003613698v1_2111797 | NW_003613794v1_2093486 |
| NW_003613604v1_2182277 | NW_003613699v1_2531401 | NW_003613796v1_1541566 |
| NW_003613605v1_2094383 | NW_003613700v1_2288776 | NW_003613797v1_115583 |
| NW_003613606v1_3016490 | NW_003613701v1_2323417 | NW_003613799v1_1734091 |
| NW_003613608v1_2694308 | NW_003613702v1_118243 | NW_003613800v1_959621 |
| NW_003613609v1_2243995 | NW_003613703v1_1906452 | NW_003613801v1_1510812 |
| NW_003613610v1_3181123 | NW_003613704v1_631098 | NW_003613803v1_903324 |
| NW_003613611v1_4102897 | NW_003613705v1_1518805 | NW_003613804v1_1994730 |
| NW_003613612v1_1652523 | NW_003613706v1_808099 | NW_003613805v1_987470 |
| NW_003613613v1_2718067 | NW_003613709v1_2453407 | NW_003613806v1_1338276 |
| NW_003613614v1_2255857 | NW_003613710v1_1028071 | NW_003613808v1_465889 |
| NW_003613615v1_2318764 | NW_003613711v1_1254033 | NW_003613810v1_1353411 |
| NW_003613616v1_56503 | NW_003613712v1_76029 | NW_003613811v1_59362 |
| NW_003613617v1_4238917 | NW_003613713v1_1280454 | NW_003613815v1_1251414 |
| | CpG Site Location | CpG Site Location |
| | NW_003613880v1_93385 | NW_003614002v1_175753 |
| | NW_003613881v1_1589164 | NW_003614004v1_1344796 |
| | NW_003613882v1_439142 | NW_003614005v1_505569 |
| | NW_003613883v1_413306 | NW_003614007v1_1157871 |
| | NW_003613884v1_378768 | NW_003614008v1_449401 |
| | NW_003613885v1_1436224 | NW_003614009v1_238441 |
| | NW_003613886v1_910244 | NW_003614010v1_1055193 |
| | NW_003613887v1_841298 | NW_003614012v1_1365366 |
| | NW_003613889v1_775996 | NW_003614013v1_95892 |
| | NW_003613890v1_1318221 | NW_003614015v1_1201448 |
| | NW_003613892v1_1268708 | NW_003614016v1_1200104 |
| | NW_003613893v1_812436 | NW_003614017v1_611729 |
| | NW_003613894v1_543971 | NW_003614018v1_942931 |
| | NW_003613896v1_1659345 | NW_003614019v1_114734 |
| | NW_003613898v1_444719 | NW_003614020v1_572928 |
| | NW_003613899v1_1676351 | NW_003614021v1_456252 |
| | NW_003613900v1_649642 | NW_003614027v1_1190182 |
| | NW_003613901v1_1509601 | NW_003614028v1_892555 |
| | NW_003613902v1_15541 | NW_003614029v1_1345266 |
| | NW_003613903v1_119318 | NW_003614030v1_626394 |
| | NW_003613904v1_1048733 | NW_003614031v1_395537 |
| | NW_003613906v1_1388010 | NW_003614033v1_1040840 |
| | NW_003613908v1_1064955 | NW_003614036v1_125704 |
| | NW_003613911v1_1033666 | NW_003614037v1_1265549 |
| | NW_003613912v1_177411 | NW_003614039v1_398115 |
| | NW_003613913v1_976964 | NW_003614040v1_1033846 |
| | NW_003613916v1_1172275 | NW_003614042v1_1015335 |
| | NW_003613918v1_1523956 | NW_003614043v1_313167 |
| | NW_003613919v1_787542 | NW_003614046v1_442298 |
| | NW_003613921v1_1426218 | NW_003614047v1_1229009 |
| | NW_003613923v1_664550 | NW_003614048v1_266917 |
| | NW_003613925v1_336232 | NW_003614050v1_1165528 |
| | NW_003613926v1_1583695 | NW_003614051v1_4205 |
| | NW_003613927v1_34206 | NW_003614052v1_1160942 |
| | NW_003613928v1_1094613 | NW_003614053v1_1066149 |
| | NW_003613930v1_1319020 | NW_003614055v1_929580 |

TABLE 7
Representative crayfish CpG sites on the multispecies DNA-methylation bead array
chip. Table shows an example 516 of the total 10741 crayfish CpG sites.

| CpG Site Location | CpG Site Location | CpG Site Location | CpG Site Location | CpG Site Location |
|---|---|---|---|---|
| SckamPh_3_77682 | SckamPh_2327_42711 | SckamPh_4782_3432 | SckamPh_8056_32549 | SckamPh_15467_5681 |
| SckamPh_9_10075351 | SckamPh_2340_200378 | SckamPh_4845_4225 | SckamPh_8067_16457 | SckamPh_15875_12442 |
| SckamPh_69_94922 | SckamPh_2343_86599 | SckamPh_4893_501 | SckamPh_8104_53433 | SckamPh_16003_12618 |
| SckamPh_114_134495 | SckamPh_2363_9227 | SckamPh_4946_38913620 | SckamPh_8216_26788 | SckamPh_16082_8013 |
| SckamPh_115_78664 | SckamPh_2394_56668 | SckamPh_4948_43589 | SckamPh_8243_37088 | SckamPh_16188_34421 |
| SckamPh_142_71359 | SckamPh_2396_47020 | SckamPh_5028_42532 | SckamPh_8311_35908 | SckamPh_16377_10276 |
| SckamPh_161_33592 | SckamPh_2471_51347 | SckamPh_5034_1502 | SckamPh_8315_843 | SckamPh_16833_3675 |
| SckamPh_212_112730 | SckamPh_2479_5143 | SckamPh_5178_42327 | SckamPh_8475_28940 | SckamPh_16968_10488 |
| SckamPh_231_379063 | SckamPh_2492_32654 | SckamPh_5182_45087 | SckamPh_8498_39377 | SckamPh_17242_82463 |
| SckamPh_325_146488 | SckamPh_2521_152617 | SckamPh_5203_13032 | SckamPh_8507_11781993 | SckamPh_17869_669 |
| SckamPh_337_2074389 | SckamPh_2564_9955 | SckamPh_5212_33517 | SckamPh_8555_10220 | SckamPh_17982_4476 |
| SckamPh_349_46948 | SckamPh_2631_31392 | SckamPh_5250_8755 | SckamPh_8679_52336 | SckamPh_18427_28662 |
| SckamPh_385_32575 | SckamPh_2634_765 | SckamPh_5254_31593 | SckamPh_8705_6373 | SckamPh_18489_4463 |
| SckamPh_391_4805 | SckamPh_2640_28710 | SckamPh_5267_10981 | SckamPh_8786_10734 | SckamPh_18503_6096 |
| SckamPh_411_3706206 | SckamPh_2668_33376 | SckamPh_5279_11023 | SckamPh_8798_779 | SckamPh_19007_5763 |
| SckamPh_487_59591 | SckamPh_2679_89014 | SckamPh_5329_23479 | SckamPh_8910_8472 | SckamPh_19271_2336 |
| SckamPh_504_48786 | SckamPh_2687_38090 | SckamPh_5343_71286 | SckamPh_9146_41919 | SckamPh_19289_17852 |
| SckamPh_543_18626441 | SckamPh_2759_24385 | SckamPh_5409_3707 | SckamPh_9180_14718 | SckamPh_19882_11057 |
| SckamPh_656_59029 | SckamPh_2817_35520 | SckamPh_5451_1205580 | SckamPh_9202_23675229 | SckamPh_20001_1926 |
| SckamPh_672_44457 | SckamPh_2819_108054 | SckamPh_5487_24040 | SckamPh_9300_21945 | SckamPh_20360_16533 |
| SckamPh_676_53349 | SckamPh_2846_66684 | SckamPh_5533_153207 | SckamPh_9318_47939 | SckamPh_20959_16252 |
| SckamPh_683_124920 | SckamPh_2860_39755 | SckamPh_5556_42221 | SckamPh_9331_17133 | SckamPh_21169_24002 |
| SckamPh_690_13322770 | SckamPh_2982_5714 | SckamPh_5641_6342034 | SckamPh_9368_35788 | SckamPh_21300_20277 |
| SckamPh_710_68582 | SckamPh_3001_36852 | SckamPh_5646_4447 | SckamPh_9436_10636 | SckamPh_21327_1866017 |
| SckamPh_727_46002 | SckamPh_3034_40966 | SckamPh_5653_50566 | SckamPh_9479_6464 | SckamPh_21484_15923 |
| SckamPh_738_303251 | SckamPh_3052_153355 | SckamPh_5680_1885063 | SckamPh_9518_5317 | SckamPh_21550_6551 |
| SckamPh_740_106211 | SckamPh_3058_101066 | SckamPh_5719_21777 | SckamPh_9585_40099 | SckamPh_21694_52183 |
| SckamPh_748_89288 | SckamPh_3071_35704 | SckamPh_5740_5705 | SckamPh_9594_4451 | SckamPh_21877_24219 |
| SckamPh_765_274245 | SckamPh_3116_36302 | SckamPh_5848_40840 | SckamPh_9631_18394868 | SckamPh_22166_15113 |
| SckamPh_817_59075 | SckamPh_3124_50650 | SckamPh_5873_15975 | SckamPh_9636_6240 | SckamPh_22355_14478 |
| SckamPh_844_63272 | SckamPh_3164_57089 | SckamPh_5889_28603 | SckamPh_9655_41432 | SckamPh_22376_106764 |
| SckamPh_858_59538 | SckamPh_3168_59862 | SckamPh_5919_47198 | SckamPh_9673_20485 | SckamPh_22383_18564 |
| SckamPh_892_2783 | SckamPh_3171_13015 | SckamPh_5994_15260 | SckamPh_9795_120858 | SckamPh_22809_23462 |
| SckamPh_938_27799 | SckamPh_3184_48846 | SckamPh_5997_5093279 | SckamPh_9824_5826 | SckamPh_22826_5819 |
| SckamPh_1000_161843 | SckamPh_3188_8374 | SckamPh_6042_25772 | SckamPh_9915_56433 | SckamPh_22829_23892 |
| SckamPh_1009_81145 | SckamPh_3197_1897 | SckamPh_6115_165162 | SckamPh_9915_56439 | SckamPh_23047_3695 |

### Example 2

A customizable methylation bead BeadChip array (Illumina) was designed for functionality with three distinct animal species: Chinese Hamster Ovary cell lines (CHO), chicken and crayfish.

The end design contained nearly 80.000 different bead types in each array. For Infinium I chemistry two bead types were used to analyze a single DNA-Methylation site. Infinium Il requires only one bead type. Due to the need of covering many DNA-methylation sites with Infinium I chemistry, the number of bead types differed from the number of analyzable DNA Methylation sites. For designing the array, the candidate CpG sites for each species was identified using the following methods for each category (Table 8).

TABLE 8
Breakdown of CpG site categories per species in
the multispecies methylation bead-array design.

| | Final DNA-Methylation sites on the |
|---|---|
| Species | TALOS chip |
| Chicken | 28083 |
| CHO | 22598 |
| Crayfish | 9023 |
| Total | 59704 |

### Example 3

### Evaluation of Methylation Values for Titrated Chicken Samples

To assess the accuracy of methylation levels as detected on the BeadChip of Example 2, DNA methylation controls were obtained from an external vendor, wherein genomic DNA samples were completely methylated and de-methylated from an off-the-shelf chicken genomic DNA sample to give 0% and 100% methylated DNA samples. These methylation controls were then further tested according to the Bisulfite conversion, BeadChip analysis and data processing sections below. The samples were run as technical triplicates across three BeadChips of Example 2.

### DNA Extraction

DNA is extracted using the PureLink Genomic DNA Isolation Minikit kit (Invitrogen), including RNAase treatment following the manufacturer's instructions. DNA quantity is measured by PicoGreen assay and DNA quality is assessed via NanoDrop (Thermo Scientific) to ensure the A260/280 ratio is ≤1.8. A small amount of sample is then also analysed using automated electrophoresis on TapeStation (Agilent) to ensure each sample contains high molecular weight DNA.

### Bisulfite Conversion and BeadChip Analysis

The genomic DNA samples are then subjected to bisulfite conversion using the EZ DNA Methylation-Gold™ Kit (Zymo Research). The methylation levels are then quantified using our customized methylation BeadChip kits (Illumina) which can analyze over 50,000 methylation sites quantitatively across the genome at single-nucleotide resolution. After bisulfite conversion, samples were processed through a three-day workflow including sample amplification, fragmentation, precipitation, hybridization to BeadChip and X-stain according to Infinium HD Methylation Assay (Illumina, Document #15019519 v07), before being imaged on the iScan (Illumina) where intensity files for the computation of beta values are generated.

### Data Processing:

The customized chip array data processing is performed in R version 4.1.2 using sesame version 1.14.2. DNA methylation level for each site was calculated as methylation B-value. Beta values are defined as methylated signal/(methylated signal+unmethylated signal). The SeSAMe pipeline (Zhou et al. 2018) was used to generate normalized B-values and for quality control. The pipeline first infers Infinium 1 channel, followed by dye Bias Correction, the Low intensity-based detection calling and making (based on p-value) was done with pOOBAH. Background subtraction based on normal-exponential deconvolution using out-of-band probes noob (Triche et al. 2013) and optionally with extra bleed-through subtraction were also implemented. After obtaining the beta values, control probes were filtered out of the data frame. CpG sites with NA beta values were also removed from the data.

### Results

The average mean values for each chip at each titration level were obtained by first calculating the average beta value for each probe. Once the average beta value per probe was obtained, the average of the all probes was calculated for that titration level.

The density plots were created by using the geom_density_ridges_gradient function from the package—‘ggridges’. The mean beta value of each probe were plotted.

Table 9 shows the methylation values for titrated samples for chicken. FIG. 2 shows the distribution plot of the mean beta value for the samples. Samples with specified methylation levels—0% and 100% were run as technical replicates (×3) across 3 chips. The mean and median β-values for each probe was computed across the 3 technical replicates within array

TABLE 9
Methylation values for titrated samples for chicken.
Mean beta values

| Chip No | 1 | 2 | 3 | |
|---|---|---|---|---|
| Chicken 0% | 0.075 | 0.067 | 0.066 | Expected 0-10% |
| Chicken 100% | 0.884 | 0.892 | 0.892 | Expected 80-100% |

### Example 4

### Evaluation of Methylation Values for Titrated CHO Samples

To assess the accuracy of methylation levels as detected on the BeadChip, genomic DNA samples from CHO-K1, DXB11, and DG44 were provided to an external vendor for complete methylation and de-methylation, then mixed in specific ratios to give 0%, 50%, 75% and 100% methylated DNA samples. These methylation controls were then further tested according to the Bisulfite conversion, BeadChip analysis and data processing sections below. The samples were run as technical triplicates across three BeadChips.

DNA extraction, bisulphite conversion, BeadChip analysis, quality control, data processing and differential methylation analysis are as outlined in Example 3.

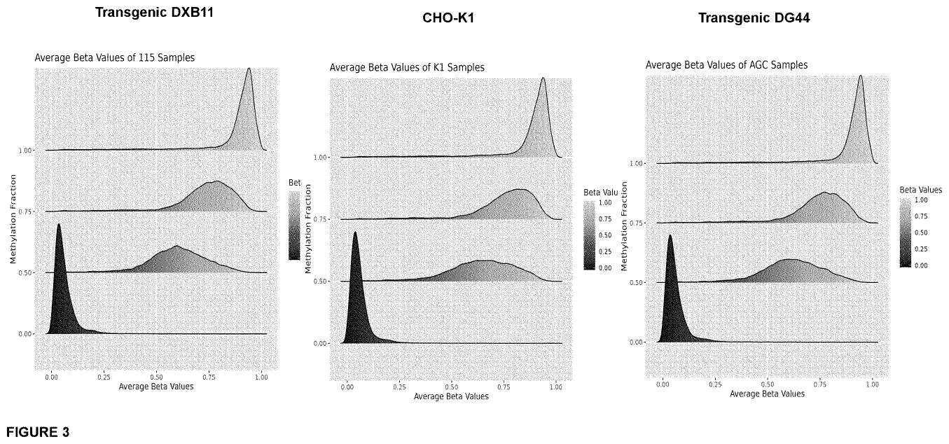

FIG. 3 shows the distribution plot of the mean beta value for the samples. Samples with specified methylation levels-0%-100% were run as technical replicates (×3) across 3 chips. The mean and median β-values for each probe was computed across the 3 technical replicates within array.

TABLE 10
methylation values for titrated samples for CHO
samples (CHO-K1, Transgenic DXB11, Transgenic DG44).
Mean beta values

| | Transgenic | | Transgenic | |
|---|---|---|---|---|
| Samples | DXB11 | CHO-K1 | DG44 | |
| CHO 0% | 0.059 | 0.058 | 0.059 | Expected 0-10% |
| CHO 50% | 0.597 | 0.639 | 0.612 | Expected 50-70% |
| CHO 75% | 0.731 | 0.753 | 0.737 | Expected 60-80% |
| CHO 100% | 0.872 | 0.868 | 0.877 | Expected |
| | | | | 80%-100% |

1. A DNA methylation-based array comprising at least:
a first plurality of distinct locations, each location having at least one probe molecule comprising a nucleic acid sequence complementary to a CpG site from a first plurality of CpG sites of a first animal species; and
a second plurality of distinct locations, each location having at least one probe molecule comprising a nucleic acid sequence complementary to a CpG site from a second plurality of CpG sites of a second animal species,
wherein the first and second animal species are each independently selected from the group consisting of virus, mammals, birds and aquatic animals, and
the mammal is at least one livestock or animal cell line;
the bird is at least one poultry; and
the aquatic animal is at least one crustacean, cephalopod or fish,
and
wherein the first plurality of CpG sites comprises at least 1000 CpG sites of the first animal species; and
the second plurality of CpG sites comprises at least 1000 CpG sites of the second animal species.

2. The array according to claim 1, wherein:
the livestock is selected from the group consisting of cow, goat, sheep, pig, horse, donkey, rabbit and mule;
the poultry is selected from the group consisting of chicken, turkey, duck, goose, and quail;
the crustacean is at least one decapod, preferably crayfish and/or shrimp and the cephalopod is at least one octopus and/or squid; and/or
the animal cell line is Chinese Hamster Ovary cell line (CHO).

3. The array according to either claim 1, wherein the array is a bead-based array.

4. The array according to claim 1, further comprising:
at least one probe molecule specific for at least one single nucleotide polymorphism (SNP) of the first species of animal; and
at least one probe molecule specific for at least one SNP of the second species of animal.

5. The array according to claim 1, wherein the first and second animal species is selected from the group consisting of salmon, shrimp, swine, chicken, crayfish, CHO, and at least one virus.

6. The array according to claim 1, wherein at least a part of the plurality of the CpG sites of the first, and second species are dynamic CpG sites.

7. The array according to claim 1, wherein the array comprises at least a third plurality of distinct locations, each location having at least one probe molecule comprising a nucleic acid sequence complementary to a CpG site from a third plurality of CpG sites of a third animal species.

8. The array according to claim 7, wherein the three animal species are chicken, crayfish and CHO cell.

9. The array according to claim 8, wherein:
(a) the first animal species is chicken and the plurality of CpG sites comprises at least:
dynamic CpG sites, CpG sites of promoters, and/or CpG sites in Low Methylated Regions (LMRs) and the CpG sites comprises at least CpG sites selected from Tables 2, 3 and 4 respectively;
(b) the second animal species is crayfish and the plurality of CpG sites comprises at least:
dynamic CpG sites, CpG sites found in methylated repeats in the crayfish genome, and/or CpG sites in immune system linked genes, meiosis genes, and DNMT1 and the CpG sites and the CpG sites comprises at least CpG sites selected from Table 7; and/or
(c) the third animal species is a CHO cell and the plurality of CpG sites comprises at least:
dynamic CpG sites, and CpG sites found in promoters, metabolic linked genes, protein production linked genes, cell growth and division linked genes, methylation linked genes, and viral promoters and the CpG sites comprises at least CpG sites selected from Tables 5 and 6.

10. A method of using the array according to claim 1, comprising predicting the biological age of a test animal with the array.

11. A method of using the array according to claim 1, comprising determining with the array if a test animal and/or a test animal from which a product is derived has been treated and/or is currently undergoing treatment with at least one antibiotic and/or veterinary chemical.

12. A method of using the array according to claim 1, comprising determining with the array a distinct certification of a test animal-derived product sample.

13. The method according to claim 12, wherein the distinct certification of the animal derived product sample is based on
(a) whether the animal has been slaughtered by a single cut across the throat severing both carotid arteries, both jugular veins, both vagus nerves, the trachea and the esophagus and/or of the test animal having been bled to death; or
(b) a type of animal husbandry that the test animal was reared under.

14. A method of using the array according to claim 1, comprising identifying with the array the geographic origin of a test animal-derived product.
